(19) United States
(12) Patent Application Publication
(10) Pub. No.: US 2012/0142645 A1
(43) Pub. Date: Jun. 7, 2012

(54) NEUROACTIVE STEROID COMPOSITIONS AND METHODS OF USE FOR LOWERING CHOLESTEROL

(76) Inventor: Christine E. Marx, Durham, NC (US)

(21) Appl. No.: 13/254,074

(22) PCT Filed: Mar. 17, 2010
(86) PCT No.: PCT/US10/27673

§ 371 Date: Feb. 20, 2012

Related U.S. Application Data
(60) Provisional application No. 61/160,834, filed on Mar. 17, 2009.

(51) Int. Cl. A61K 31/56; A61P 7/00; A61P 3/06; A61K 31/566

(52) U.S. Cl. 514/170

(57) ABSTRACT

Methods for ameliorating a symptom associated with hypercholesterolemia, hyperlipidemia, or both in a subject. In some embodiments, the methods include administering to a subject in need thereof an effective amount of a composition comprising pregnenolone (PG), allopregnanolone (ALLO), dehydroepiandrosterone (DHEA), progesterone (PROG), precursors thereof, metabolites thereof, pharmaceutically acceptable salts thereof, derivatives thereof, or combinations thereof. Also provided are methods for ameliorating at least one symptom resulting from undesirable cholesterol biosynthesis in a subject and methods for lowering cholesterol, low density lipoprotein, or both in the serum of a subject.

### RELATED APPLICATION

This application claims priority to U.S. Provisional Application Ser. No. 61/160,834, filed Mar. 17, 2009, the disclosure of which is incorporated herein by reference in its entirety.

### TECHNICAL FIELD

The presently disclosed subject matter relates to methods for treating hypercholesterolemia and/or ameliorating one or more symptoms thereof, comprising administering to a subject in need thereof an effective amount of one or more neuroactive steroids, pharmaceutically acceptable salts thereof, derivatives thereof, precursors thereof, metabolites thereof, or combinations thereof. Also provided are methods for prophylaxis comprising administering to a subject in need thereof one of the presently disclosed neuroactive steroids, pharmaceutically acceptable salts thereof, derivatives thereof, precursors thereof, metabolites thereof, or combinations thereof.

### BACKGROUND

Atherosclerosis is a progressive disease characterized by the thickening and/or hardening and loss of elasticity of inner artery walls. The pathologic process likely is responsible for most coronary heart disease (CHD) and strokes.

Since atherosclerosis is a leading cause of mortality and morbidity in the world, intense research efforts have been dedicated to the disease. For example, it was proposed that high dietary cholesterol intake was associated with the development of atherosclerosis as early as 1913 (Anitschkow & Chalatov, 1913). Consequently, lipid-lowering therapy has been a major approach in attempts to prevent and/or treat atherosclerosis-related CHD and stroke. This therapeutic method treats the elevated level of low-density lipoprotein (LDL) or cholesterol in blood as a primary cause in atherosclerosis (Grundy, 1992).

In deciding whether a particular subject is in need of therapy to prevent or to treat the disease, physicians usually rely heavily on measuring the LDL concentration in the subject's blood. The expert panels in the United States of America, Europe, the United Kingdom, and Canada have defined the guidelines of LDL level in serum (Shephered et al., 1987; Study Group of the European Atherosclerosis Society, 1988; Canadian Lipoprotein Conference Ad Hoc Committee on Guidelines for Dyslipoproteinemias, 1990; National Cholesterol Education Program, 2002).

Epidemiological studies have suggested that many risk factors, including elevated LDL levels, hypertension, cigarette smoking, family history, systemic inflammation such as rheumatoid arthritis, infectious agents such as Chlamydia pneumoniae, a high-fat diet, and psychological factors such as depression, are associated with increased risk of atherosclerosis. However, current screening methods are not adequate to predict which individuals are likely to develop atherosclerosis.

Typically, cholesterol is carried in the blood of warm-blooded animals in certain lipid-protein complexes such as chylomicrons, very low density lipoprotein (VLDL), low density lipoprotein (LDL), and high density lipoprotein (HDL). LDL appears to function in a way that directly results in deposition of the LDL cholesterol in the blood-vessel wall and that HDL functions in a way that results in the HDL picking up cholesterol from the vessel wall and transporting it to the liver where it is metabolized (Miller, 1980; Brown & Goldstein, 1983). For example, in various epidemiologic studies the LDL cholesterol levels correlate well with the risk of coronary heart disease whereas the HDL cholesterol levels are inversely associated with coronary heart disease (see e.g., Mao et al., 1983; Patton et al., 1983). It is generally accepted that reduction of abnormally high LDL cholesterol levels is effective therapy not only in the treatment of hypercholesterolemia but also in the treatment of atherosclerosis.

Therefore, there exists a long-felt and ongoing need in the art for improved methods and new compositions for treating subjects with symptoms associated with abnormal and/or undesirable cholesterol biosysthesis. Also urgently needed are new methods and compositions for treating subjects prophylactically who might be at risk for developing one or more symptoms typically associated with hypercholesterolemia and/or hyperlipidemia.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides methods for ameliorating a symptom associated with hypercholesterolemia, hyperlipidemia, or both in a subject.

The presently disclosed subject matter also provides methods for ameliorating at least one symptom resulting from undesirable cholesterol biosynthesis in a subject. In some embodiments, the undesirable cholesterol biosynthesis in subject results in hypercholesterolemia, hyperlipidemia, or a combination thereof in the subject.

The presently disclosed subject matter also provides methods for lowering cholesterol, low density lipoprotein, or both in the serum of a subject.

In some embodiments, the presently disclosed methods comprise administering to a subject in need thereof an effective amount of a composition comprising pregnenolone (PG), allopregnanolone (ALLO), dehydroepiandrosterone (DHEA), progesterone (PROG), precursors thereof, metabolites thereof, pharmaceutically acceptable salts thereof, derivatives thereof, or combinations thereof. In some embodiments, the composition comprises at least two active agents selected from the group consisting of PG, ALLO, DHEA, PROG, precursors thereof, metabolites thereof, pharmaceutically acceptable salts thereof, derivatives thereof, or combinations thereof. In some embodiments, the effective amount is sufficient to lower blood cholesterol, lower blood low density lipoprotein (LDL), raise blood high density lipoprotein (HDL), or combinations thereof in the subject. In some embodiments, the derivative comprises a sulfated derivative. In some embodiments of the presently disclosed methods, the administering causes substantially no decrease in high density lipoprotein (HDL) levels in the blood of the subject.

In some embodiments of the presently disclosed methods, the composition is administered in a sustained release formulation, a controlled release formulation, or a combination thereof. In some embodiments, the sustained release formulation, the controlled release formulation, or the combination thereof is selected from the group consisting of an oral formulation, a peroral formulation, a buccal formulation, an enteral formulation, a pulmonary formulation, a rectal formulation, a vaginal formulation, a nasal formulation, a lingual formulation, a sublingual formulation, an intravenous formulation, an intraarterial formulation, an intracardial formulation, an intramuscular formulation, an intraperitoneal formulation, a transdermal formulation, an intracranial formulation, an intracutaneous formulation, a subcutaneous formulation, an aerosolized formulation, an ocular formulation, an implantable formulation, a depot injection formulation, and combinations thereof. In some embodiments of the presently disclosed methods, the composition comprises PG, a sulfated derivative of PG, a precursor of PG, a metabolite of PG, a pharmaceutically acceptable salt of PG, or a combination thereof.

In some embodiments, the presently disclosed methods further comprise administering to the subject at least one additional cholesterol lowering composition, wherein the at least one additional cholesterol lowering composition is administered to the subject before, after, and/or at the same time as the composition comprising PG, ALLO, DHEA, PROG, precursors thereof, metabolites thereof, pharmaceutically acceptable salts thereof, derivatives thereof, or combinations thereof.

In some embodiments, the presently disclosed methods further comprise administering to the subject an additional pharmaceutical composition comprising an active agent selected from the group consisting of a cholesterol lowering agent, an LDL reducing agent, an HDL increasing agent, or a combination thereof.

In some embodiments of the presently disclosed methods, the subject also has a neuropsychiatric disorder. In some embodiments, the neuropsychiatric disorder is selected from the group consisting of a psychotic disorder, a cognitive disorder, a neurodegenerative disorder, an anxiety disorder, a pain disorder, and combinations thereof. In some embodiments, the neuropsychiatric disorder is selected from the group consisting of schizophrenia, schizoaffective disorder, Alzheimer's disease, Attention Deficit Disorder/Attention Deficit Hyperactivity Disorder, depression, bipolar disorder, post-traumatic stress disorder (PTSD), alcohol abuse, alcohol dependence, drug dependence, drug abuse, and combinations thereof.

Thus, the presently disclosed subject matter provides in some embodiments methods for ameliorating a symptom associated with hypercholesterolemia, hyperlipidemia, or both in a subject.

An object of the presently disclosed subject matter having been stated hereinabove, and which is achieved in whole or in part by the presently disclosed subject matter, other objects will become evident as the description proceeds.

### BRIEF DESCRIPTION OF THE FIGURES

FIGS. 1A-1C are a series of graphs showing associations between neurosteroid levels in subjects and improvements in cognitive performance.

FIG. 1A is a graph showing that increases in serum pregnenolone following treatment with this neurosteroid were correlated with improvements in cognitive performance, as assessed by composite BAGS z-scores.

FIG. 1B is a graph showing that increases in serum allopregnanolone following treatment with pregnenolone are correlated with cognitive improvement, as assessed by composite BAGS z-scores.

FIG. 1C is a graph showing that baseline pregnenolone sulfate (PGS) levels are inversely associated with cognitive improvement, as assessed by composite Measurement and Treatment Researfch to Improve Cognition in Schizophrenia (MATRICS, also referred to as MCCB) t-scores.

FIGS. 2A and 2B are schematic diagrams depicting the biosynthetic pathways by which cholesterol is synthesized from acetyl-CoA (FIG. 2A) and by which neurosteroids are synthesized from cholesterol (FIG. 2B).

FIG. 3 is a plot showing that total cholesterol levels were reduced in subjects with mild traumatic brain injury (TBI) following treatment with pregnenolone versus placebo. Each square corresponds to an individual subject with mild TBI.

### DETAILED DESCRIPTION

### I. General Considerations

Neurosteroids (sometimes referred to as “neuroactive steroids” are modulators of neurotransmitter receptors and other receptors in the brain. They are generally synthesized in the brain and other areas of the central and peripheral nervous systems from cholesterol and other precursors (Mellon & Griffin, 2002; Agis-Balboa et al., 2006). Particularly, neuroactive steroids can be synthesized in the brain (neurosteroids), adrenals, or gonads, and can rapidly alter neuronal excitability by acting at ligand-gated ion channel receptors, including NMDA and GABA_{A} receptors (Paul & Purdy, 1992; Rupprecht & Holsboer, 1999). For example, DHEA and PGS are positive modulators of excitatory NMDA receptors (Irwin et al., 1994; Wu et al., 1991; Compagnone & Mellon, 1998; Debonnel et al., 1996) and negative modulators of inhibitory GABA_{A} receptors (Majewska et al., 1988; Imamura & Prasad, 1998; Park-Chung et al., 1999). Conversely, neuroactive steroids such as ALLO are positive modulators of GABA_{A} receptors, potentiating GABA_{A} receptor response more potently than benzodiazepines or barbiturates (Majewska et al., 1986; Morrow et al., 1987, Morrow et al., 1990). ALLO increases with a number of acute stressors in rodent models (Purdy et al., 1991; Barbaccia et al., 1996, Barbaccia et al., 1998; Morrow et al., 1995; Vallee et al., 2000), and might represent a component of an endogenous regulatory mechanism that contributes to the modulation of hypothalamic-pituitary-adrenal (HPA) axis activity (Morrow et al., 1995). The HPA axis might be relevant to the pathophysiology of nicotine dependence, and rodent evidence suggests that nicotine administration dose-dependently increases ALLO and PG levels (Porcu et al., 2003). Although a number of studies have investigated nicotine and the HPA axis by targeting cortisol and corticosterone in clinical and preclinical models, respectively, data are currently more limited regarding other steroids that are also produced in the adrenal (as well as other sites), including ALLO, PG, and DHEAS.

Pregnenolone (PG) is an exemplary neurosteroid. Its sulfated derivative pregnenolone sulfate (PGS) demonstrates effects at membrane-bound ligand-gated ion channel receptors such as N-methyl-D-aspartic acid (NMDA) receptors. PG and PGS enhance learning and memory in rodent models (Vallee et al., 1997; Vallee et al., 2000; Vallee et al., 2001; Akwa et al., 2001; Flood et al., 1992; Flood et al., 1995). These effects might be NMDA receptor-mediated. PGS also increases acetylcholine release in rodent hippocampus and cortex, and these actions represent another potential mechanism for its effects on learning and memory in rodent models. Other positive modulators of NMDA receptors (including glycine, serine, and D-cycloserine) might decrease negative symptoms in patients with schizophrenia (paucity of speech, avolition, anhedonia, affective flattening, etc.), and might also impact cognitive symptoms. PG is also elevated following certain antipsychotic agents and may contribute to their therapeutic efficacy (Marx et al., 2006a; Marx et al., 2006d)

Cognitive symptoms and negative symptoms in patients with schizophrenia are frequently severe, and strongly correlated with decreased functional outcome and quality of life. NMDA receptors are known to impact learning and memory. Cognitive deficits have been associated with poor treatment outcomes in subjects with certain neuropsychiatric disorders (NPDs) including, but not limited to schizophrenia. PG has been investigated for the treatment of rheumatoid arthritis and other disorders in humans, and shown to be safe, well-tolerated.

Subjects with NPDs (e.g., schizophrenia and schizoaffective disorder) frequently demonstrate significant cognitive deficits, and these deficits are more closely related to functional outcome than any other symptom domain (including “positive symptoms” such as auditory hallucinations and delusions). The newer antipsychotics (also referred to as “second-generation” or “atypical” antipsychotics) have only modest effects on cognitive outcomes. These newer agents do not appear to further impair cognitive functioning, however, a side effect frequently attributed to the older antipsychotics (also designated “conventional”, “first-generation”, or “typical” antipsychotics). The improved side effect profiles of the newer agents with regard to cognitive functioning represent progress, but effective agents to improve cognitive symptoms in NPDs such as schizophrenia and schizoaffective disorder still represent an urgent clinical need. Furthermore, a number of second generation antipsychotic agents have been associated with increased risk for weight gain, diabetes, and dyslipidemias. Thus, new agents with improved side effect profiles are needed.

Other evidence suggests that NMDA antagonists such as ketamine induce psychotic symptoms. Positive modulation of NMDA receptors might therefore improve symptoms of NPDs. Several studies demonstrating that agonists of the glycine modulatory site of the NMDA receptor may improve negative symptoms in some patients support this possibility (Goff et al., 1999a; Heresco-Levy et al., 1999).

In recent years, the impact of cognitive deficits on patient functioning has been recognized (Green, 1996), and investigations into the amelioration of cognitive deficits found in NPDs have received increasing attention. It is possible that other positive modulators of NMDA receptors may have efficacy for neurocognitive symptoms in schizophrenia and other NPDs. Since the sulfated derivative of PG is a positive modulator of NMDA receptors and increases acetylcholine release in rodent hippocampus, PG, its derivatives, and/or its metabolites might be helpful for cognitive symptoms in subjects with NPDs. In addition, second-generation antipsychotics such as clozapine and olanzapine elevate PG levels in rodent hippocampus to concentrations that are physiologically relevant. If antipsychotics also elevate PG in subjects with psychosis, it is possible that elevations in PG and other neuroactive steroids might contribute to antipsychotic therapeutic efficacy.

Cognitive and negative symptoms in schizophrenia are frequently severe and strongly correlated with decreased functional outcome and quality of life (Green et al., 2000; Harvey et al., 2004; Marder & Fenton, 2004; Buchanan et al., 2005; Buchanan, 2006; Green, 2006; Kirkpatrick et al., 2006; Marder, 2006), but effective pharmacological interventions remain limited for these symptom domains (Buchanan et al., 2005; Kirkpatrick et al., 2006; Buchanan, 2007; Buchanan et al., 2007; Carter et al., 2008). Although first- and second-generation antipsychotic agents have been reported to produce improvements in cognitive and negative symptoms, the emerging scientific consensus appears to be that these effects are generally modest in scope, and that significant disabling symptoms frequently persist despite positive symptom reduction (Buchanan et al., 2005; Kirkpatrick et al., 2006; Keefe et al., 2007). New treatment strategies are therefore necessary to address these important dimensions of schizophrenia symptomatology.

The neurosteroid pregnenolone represents a promising agent for cognitive and negative symptoms in schizophrenia. Pregnenolone (Flood et al., 1992) and its sulfated derivative, pregnenolone sulfate (Mayo et al., 1993; Flood et al., 1995; Meziane et al., 1996; Vallee et al., 1997; Pallares et al., 1998; Ladurelle et al., 2000; Akwa et al., 2001; Vallee et al., 2001; Darnaudery et al., 2002) enhance learning and memory in animal models. Pregnenolone sulfate positively modulates glutamatergic N-methyl-D-aspartate (NMDA) receptors (Wu et al., 1991; Bowlby, 1993; Irwin et al., 1994), enhances long-term potentiation in rat hippocampus (Sliwinski et al., 2004), and prevents learning and memory deficits induced by NMDA receptor antagonists, including MK-801 and DAP-V (Mathis et al., 1994; Romeo et al., 1994; Cheney et al., 1995; Mathis et al., 1996; Akwa et al., 2001). Treatment with pregnenolone, which is metabolized to pregnenolone sulfate in humans following acute administration (Roberts, 1995; Morley et al., 1997), thus represents a promising strategy for the amelioration of hypothesized NMDA receptor hypofunction in schizophrenia (Javitt, 2004; Millan, 2005; Coyle, 2006; Rujescu et al., 2006; Javitt, 2007). Pregnenolone sulfate also increases acetylcholine release in multiple rodent brain regions (Mayo et al., 1993; Vallee et al., 1997; Darnaudery et al., 1998; Pallares et al., 1998; Darnaudery et al., 2002) and prevents the memory-impairing effects of the muscarinic cholinergic receptor antagonist scopolamine (Meziane et al., 1996; Vallee et al., 2001), actions consistent with cognitive enhancement. In addition, clozapine markedly increases pregnenolone levels in rodent hippocampus, representing a candidate mechanism for its superior efficacy (Marx et al., 2006a). Converging evidence thus suggests that pregnenolone might have potential utility for the treatment of cognitive and negative symptoms in schizophrenia.

Despite the availability of pregnenolone as a dietary supplement in the United States, very few clinical trials have investigated its use. Studies conducted in the 1940s and 1950s showed that pregnenolone 25-500 mg/day was safe and well-tolerated in humans (Pincus & Hoagland, 1944; Pincus and Hoagland, 1945a; Pincus and Hoagland, 1945b; Davison et al., 1950; Freeman et al., 1950; Guest et al., 1950; Henderson et al., 1950; McGavack et al., 1951). A number of these older studies treated patients with inflammatory diseases such as rheumatoid arthritis, and several reported improvements in symptoms and overall functioning following pregnenolone. According to these earlier reports, pregnenolone was very well-tolerated with minimal side effects, and did not affect weight, heart rate, blood pressure (even in patients with hypertension), menstrual cycle, or glucose levels (either in diabetics or non-diabetics). In a more recent investigation, pregnenolone at low doses (15-30 mg/day) was generally well-tolerated in healthy volunteers with no significant side effects compared to placebo (Meieran et al., 2004). Evidence to date thus suggests that pregnenolone has a favorable safety profile, although controlled trials remain very limited.

Pregnenolone was studied as an adjunctive therapeutic strategy for cognitive and negative symptoms in patients with schizophrenia or schizoaffective disorder with an escalating fixed-dose approach, achieving a total dose of 500 mg/day in the last 4 weeks of the study administered in divided doses. The dosing schedule was informed by prior pregnenolone investigations that demonstrated safety and tolerability. Although human pharmacokinetic studies addressing acute administration are extremely few, a single oral dose of pregnenolone 175 mg approximately doubles serum levels over the course of 4-8 hours (Roberts, 1995). Since pregnenolone levels decrease by approximately 60% with age (Morley et al., 1997; Roberts, 1995), and since it was estimated that mean study participant age would be greater than 40, it was projected that the dosing strategy of pregnenolone 500 mg/day in the last 4 weeks of the study would likely produce pregnenolone levels that are close to those observed in young adulthood or up to 2-fold higher than typical young adult levels.

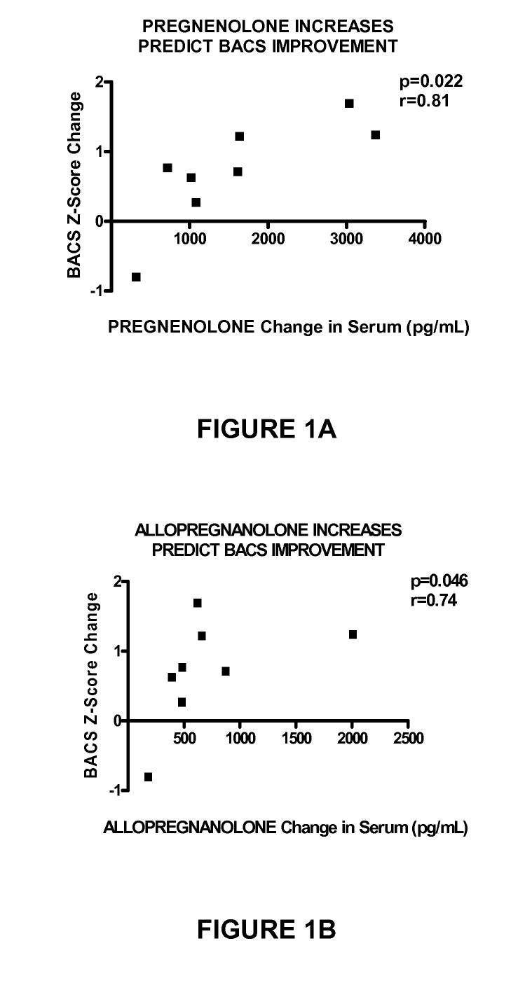

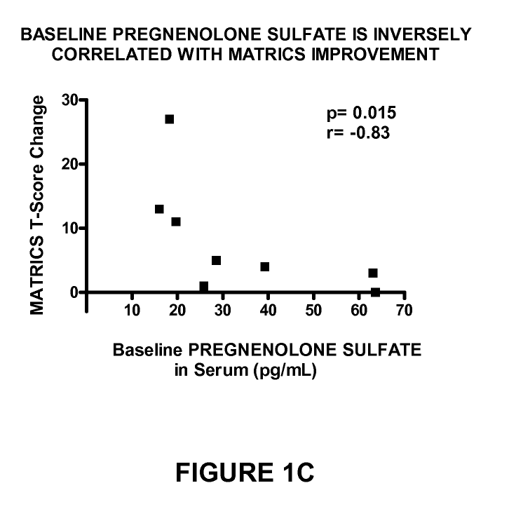

Orally administered PG appears to be well-absorbed and converted to its sulfated form (PGS; see FIG. 1). Studies in rodents have shown that both PG and PGS appear to be transported across the blood brain barrier. Thus, several studies have demonstrated that oral administration of PG is safe, well tolerated, and likely results in elevated brain levels of both PG and PGS.

The presently disclosed subject matter also relates to compositions comprising other neuroactive steroids and methods of using the same. One such neuroactive steroid is allopregnanolone (3α-hydroxy-5α-pregnan-20-one; ALLO). ALLO is synthesized de novo in the brain from cholesterol or from peripheral steroid precursors (Belelli & Lambert, 2005). A number of ALLO actions are attributed to the fact that it potentiates GABA_{A} receptor responses at nanomolar concentrations, doing so more potently than either benzodiazepines or barbiturates (Morrow et al., 1987). ALLO demonstrates anxiolytic (Wieland et al., 1991) and anticonvulsant effects (Kokate et al., 1996).

More recently, neuroprotective roles for ALLO have been demonstrated in a mouse model of Niemann-Pick type C disease (Griffin et al., 2004) and a rat model of traumatic brain injury (Djebaili et al., 2005). ALLO also protects against apoptosis via Bcl-2 protein in rat adrenal chromaffin and pheochromocytoma cells (Charalampopoulos et al., 2004) and protects against N-methyl-D-aspartate (NMDA)-induced apoptosis in mouse P19-derived neurons (Xilouri & Papazafiri, 2006).

As described herein, it became apparent from studies designed to test the ability of neurosteroid administration to modulate one or more symptoms associated with the conditions described hereinabove that a beneficial side effect of neurosteroid administration was a lowering of cholesterol and low density lipoprotein (LDL) in treated subjects. Thus, disclosed herein are compositions and methods for lowering cholesterol and LDL in the blood of subjects in need thereof. Also disclosed are compositions and methods for ameliorating a symptom associated with hypercholesterolemia, hyperlipidemia, or both in a subject in need thereof, and compositions and methods for ameliorating a symptom associated with undesirable cholesterol biosynthesis in a subject in need thereof.

### II. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the articles “a”, “an”, and “the” refer to “one or more” when used in this application, including in the claims. For example, the phrase “a symptom” refers to one or more symptoms. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”.

Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the phrase “biological sample” refers to a sample isolated from a subject (e.g., a biopsy) or from a cell or tissue from a subject (e.g., protein, RNA, and/or DNA isolated therefrom). Biological samples can be of any biological tissue or fluid or cells from any organism as well as cells cultured in vitro, such as cell lines and tissue culture cells. Frequently the sample will be a “clinical sample” which is a sample derived from a subject (i.e., a subject undergoing a diagnostic procedure and/or a treatment). Typical clinical samples include, but are not limited to cerebrospinal fluid, serum, plasma, blood, saliva, skin, muscle, olfactory tissue, lacrimal fluid, synovial fluid, nail tissue, hair, feces, urine, a tissue or cell type, and combinations thereof, tissue or fine needle biopsy samples, and cells therefrom. Biological samples can also include sections of tissues, such as frozen sections or formalin fixed sections taken for histological purposes.

As used herein, the term “neuropsychiatric disorder” is intended to refer broadly to any disorder of emotional, personality, and/or mental function that is of neurological origin, psychiatric origin, psychological origin, or mixed origin that negatively impacts the emotional and/or cognitive functioning of a subject. Representative neuropsychiatric disorders include those listed in the Diagnostic and Statistical Manual of Mental Disorders (DSM; including DSM-IV and DSM-IV-TR). More particularly, the term includes, but is not limited to such exemplary conditions as substance use disorders (e.g., use, abuse, and/or dependence on cocaine, opioid, cannabis, amphetamine, alcohol, caffeine, tobacco/nicotine, hallucinogens); anxiety disorders (e.g., post-traumatic stress disorder, obsessive compulsive disorder, panic disorder, agoraphobia, social phobia, acute stress disorder, generalized anxiety disorder, substance-induced anxiety disorder); mood disorders (e.g., both depressive and manic disorders including but not limited to major depressive disorder, major depressive disorder with psychotic features, major depressive disorder with postpartum onset, dysthymic disorder, bipolar I disorder, bipolar II disorder, cyclothymic disorder, substance-induced mood disorder); psychotic disorders (e.g., schizophrenia, schizoaffective disorder, delusional disorder, brief psychotic disorder, shared psychotic disorder, psychotic disorder due to a medical condition, substance-induced psychotic disorder, psychotic disorder not otherwise specified); cognitive disorders (e.g., mild cognitive impairment, Alzheimer's disease, vascular dementia, dementia due to other medical conditions, dementia due to multiple etiologies, substance-induced persisting amnestic disorder, amnestic disorder not otherwise specified, delirium). In some embodiments, the neuropsychiatric disorder is selected from the group consisting of schizophrenia, schizoaffective disorder, Alzheimer's disease, Attention Deficit Disorder/Attention Deficit Hyperactivity Disorder, depression, bipolar disorder, post-traumatic stress disorder (PTSD), a pain disorder, tobacco dependence, alcohol abuse, alcohol dependence, drug dependence, drug abuse, neurodegenerative disorders, sleep disorders, traumatic brain injury and/or concussion, and combinations thereof.

As used herein, the term “prophylaxis” and grammatical variants thereof are intended to refer to the prevention, inhibition, and/or lessening of the development of a symptom associated with hypercholesterolemia, hyperlipidemia, or both, or a symptom associated with undesirable cholesterol biosynthesis in a subject whether that symptom is already present or not. As such, “prophylaxis” is not intended to refer only to modulating the development of a symptom in a subject in which the symptom is completely absent but is also intended to refer to ameliorating the symptom in a subject in which it exists as well as preventing, inhibiting, and/or lessening any worsening of the symptom in the subject that might occur for any reason. Thus, the term “prophylaxis” is intended to overlap with yet be broader than the term “ameliorate”.

As used herein, the term “subject” refers to any organism for which diagnosis, treatment, and/or prophylaxis would be desirable. Thus, the term “subject” is desirably a human subject, although it is to be understood that the principles of the presently disclosed subject matter indicate that the presently disclosed subject matter is effective with respect to other species, including mammals, which are intended to be included in the term “subject”. Moreover, a mammal is understood to include any mammalian species for which diagnosis, treatment, and/or prophylaxis is desirable, particularly agricultural and domestic mammalian species. The methods of the presently disclosed subject matter are particularly useful in the diagnosis, treatment, and/or prophylaxis of warm-blooded vertebrates, e.g., mammals and birds.

More particularly, the presently disclosed subject matter can be used for diagnosis, treatment, and/or prophylaxis of a mammal such as a human. Also provided are methods for diagnosis, treatment, and/or prophylaxis of, and/or ameliorating a symptom associated with a neuropsychiatric disorder in, mammals of importance due to being endangered (such as Siberian tigers), of economic importance (animals raised on farms for consumption by humans) and/or social importance (animals kept as pets or in zoos) to humans.

### III. Treatment and/or Prophylaxis Methods

The presently disclosed subject matter provides methods for ameliorating a symptom associated with hypercholesterolemia, hyperlipidemia, or both in a subject. The presently disclosed subject matter also provides methods for ameliorating at least one symptom resulting from undesirable cholesterol biosynthesis in a subject. In some embodiments, the undesirable cholesterol biosynthesis in subject results in hypercholesterolemia, hyperlipidemia, or a combination thereof in the subject. The presently disclosed subject matter also provides methods for lowering cholesterol, low density lipoprotein, or both in the serum of a subject.

In some embodiments, the presently disclosed methods further comprise administering to the subject an additional pharmaceutical composition comprising an active agent selected from the group consisting of a cholesterol lowering agent, an LDL reducing agent, an HDL increasing agent, or a combination thereof.

As used herein, the phrase “ameliorating a symptom” and grammatical variants thereof refers to providing an improvement in a symptom associated with hypercholesterolemia, hyperlipidemia, or both in a subject. The improvement can be by any measure whatsoever, including a subjective assessment by the subject him or herself. Thus, the methods provided herein can ameliorate a symptom associated with hypercholesterolemia, hyperlipidemia, or both to a degree that is measurable using some clinical criterion. The nature and extent of the amelioration of the symptom associated with hypercholesterolemia, hyperlipidemia, or both does not limit the presently disclosed subject matter. One of ordinary skill in the art is familiar with methods to measure amelioration of symptoms.

As used herein, the phrase “symptom associated with hypercholesterolemia, hyperlipidemia, or both” refers to any symptom that is typically observed in a subject that has hypercholesterolemia, hyperlipidemia, or both. Representative such symptoms include, but are not limited to elevated serum cholesterol, elevated serum lipids, elevated triglycerides, and atherosclerotic plaques.

The presently disclosed subject matter also provides methods for delaying or preventing the onset of, and/or decreasing the severity of, a symptom associated with hypercholesterolemia, hyperlipidemia, or both in a subject. In some embodiments, a neuroactive steroid composition is thus administered as a therapeutic to maintain a current state of well-being of a subject with hypercholesterolemia, hyperlipidemia, or both. Thus, in some embodiments a neuroactive steroid composition of the presently disclosed subject matter is administered to a subject as a maintenance therapy to prevent the worsening of symptoms that subjects with hypercholesterolemia, hyperlipidemia, or both have and/or are at risk of developing.

In some embodiments, the subject does not have hypercholesterolemia, hyperlipidemia, or both, but is at risk for developing one or more symptoms that are associated with hypercholesterolemia, hyperlipidemia, or both, whether or not the subject develops hypercholesterolemia, hyperlipidemia, or both. The development of such symptoms can also occur as a result of other biochemical and biological alterations in the subject that are not caused by hypercholesterolemia, hyperlipidemia, or both.

In some embodiments of the presently disclosed methods, the administering causes substantially no decrease in high density lipoprotein (HDL) levels in the blood of the subject. As used herein, the term “substantially” refers to a change of in some embodiments no more than 1%, in some embodiments no more than 2%, in some embodiments no more than 3%, in some embodiments no more than 4%, in some embodiments no more than 5%, in some embodiments no more than 6%, in some embodiments no more than 7%, in some embodiments no more than 8%, in some embodiments no more than 9%, in some embodiments no more than 10%, in some embodiments no more than 15%, in some embodiments no more than 20%, and in some embodiments no more than 25% from a baseline measure (e.g., a level determined at a time prior to the administration of a composition of the presently disclosed subject matter).

In some embodiments, the presently disclosed methods further comprise administering to the subject at least one additional cholesterol lowering composition, wherein the at least one additional cholesterol lowering composition is administered to the subject before, after, and/or at the same time as the composition comprising PG, ALLO, DHEA, PROG, precursors thereof, metabolites thereof, pharmaceutically acceptable salts thereof, derivatives thereof, or combinations thereof.

Thus, the compositions and methods of the presently disclosed subject matter can be employed as part of a combination therapy in which other compositions that are known or expected to lower cholesterol (so-called “cholesterol lowering agents”) are also employed. Exemplary cholesterol lowering agents include, but are not limited to HMG-CoA reductase inhibitors; HMG-CoA synthase inhibitors; squalene epoxidase inhibitors; squalene synthetase inhibitors; y-tocotrienol, 6-tocotrienol, and tocotrienol; niacin; gemfibrozil; clofibrate; and LDL-receptor gene inducers. Alternatively or in addition, compositions that are known to lower LDL and/or raise HDL can also be employed as part of a combination therapy with the compositions and methods of the presently disclosed subject matter.

### IV. Neuroactive Steroid Compositions

The presently disclosed subject matter employs neuroactive steroid compositions comprising PG, ALLO, DHEA, pharmaceutically acceptable salts thereof, derivatives thereof, or combinations thereof. In some embodiments, the neuroactive steroid combinations comprise at least two active agents selected from the group consisting of PG, ALLO, DHEA, pharmaceutically acceptable salts thereof, and derivatives thereof. In some embodiments, the derivative comprises a sulfated derivative.

In some embodiments of the presently disclosed subject matter, the neuroactive steroid composition comprises an effective amount of PG, ALLO, DHEA, pharmaceutically acceptable salts thereof, derivatives thereof, or combinations thereof. In some embodiments, the effective amount is sufficient to raise the level of PG, ALLO, DHEA, derivatives thereof, or combinations thereof in a source selected from the group consisting of cerebrospinal fluid, serum, plasma, blood, saliva, skin, muscle, olfactory tissue, lacrimal fluid, synovial fluid, nail tissue, hair, feces, urine, in the subject by at least 1.5-fold within 8 weeks from a level in the source in the subject prior to the administering step. In some embodiments, the effective amount comprises a daily dose of at least 0.005 mg per day. In some embodiments, the effective dose comprises a dose ranging from about 0.005 mg to about 2000 mg of PG, ALLO, or DHEA, or an equivalent molar amount of the pharmaceutically acceptable salt thereof, the derivative thereof, or the combinations thereof. In some embodiments, the effective amount is sufficient to improve a cognitive function in the subject. In some embodiments, the neuroactive steroid composition comprises an effective amount of each of two or more of PG, ALLO, DHEA, derivatives thereof, or combinations thereof.

IV.A. Formulations

A neuroactive steroid composition as described herein comprises in some embodiments a composition that includes a pharmaceutically acceptable carrier. Suitable formulations include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents.

The compositions used in the methods can take such forms as suspensions, solutions, or emulsions in oily or aqueous vehicles, and can contain formulatory agents such as suspending, stabilizing, and/or dispersing agents. The compositions used in the methods can take such forms as inhalational formulations as well as oral and IV, including but not limited to fine powder formulations and droplet-generating formulations. Alternatively or in addition, the active ingredient can be in powder form for constitution with a suitable vehicle (e.g., sterile pyrogen-free water) before use.

The formulations can be presented in unit-dose or multi-dose containers, for example sealed ampules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulfate). The tablets can be coated by methods known in the art. For example, a neuroactive steroid can be formulated in combination with hydrochlorothiazide, and as a pH stabilized core having an enteric or delayed-release coating which protects the neuroactive steroid until it reaches the colon.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring, and sweetening agents as appropriate. Preparations for oral administration can be suitably formulated to give controlled release of the active compound. For buccal administration the compositions can take the form of tablets or lozenges formulated in conventional manner.

The compounds can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in rectal compositions (e.g., suppositories or retention enemas containing conventional suppository bases such as cocoa butter or other glycerides), creams or lotions, or transdermal patches.

In some embodiments, the presently disclosed subject matter employs a neuroactive steroid composition that is pharmaceutically acceptable for use in humans. One of ordinary skill in the art understands the nature of those components that can be present in a neuroactive steroid composition that is pharmaceutically acceptable for use in humans and also what components should be excluded from a neuroactive steroid composition that is pharmaceutically acceptable for use in humans.

IV.B. Doses

The term “effective amount” is used herein to refer to an amount of the therapeutic composition (e.g., a composition comprising a neuroactive steroid, a pharmaceutically acceptable salt thereof, a derivative thereof, or a combination thereof) sufficient to produce a measurable biological response (e.g., an amelioration of a symptom). Actual dosage levels of active ingredients in a neuroactive steroid composition of the presently disclosed subject matter can be varied so as to administer an amount of the active compound(s) that is effective to achieve the desired response for a particular subject and/or application. The selected dosage level can depend upon a variety of factors including the activity of the neuroactive steroid composition, formulation, route of administration, combination with other drugs or treatments, severity of the condition being treated, and physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are known to those of ordinary skill in the art.

For administration of a neuroactive steroid composition as disclosed herein, conventional methods of extrapolating human dosage based on doses administered to a murine animal model can be carried out using techniques known to one of ordinary skill in the art. Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al., 1966. Briefly, to express a mg/kg dose in any given species as the equivalent mg/m² dose, multiply the dose by the appropriate km factor. In an adult human, 100 mg/kg is equivalent to 100 mg/kg×37 kg/m²=3700 mg/m².

In some embodiments of the presently disclosed subject matter, the neuroactive steroid composition comprises an effective amount of PG, ALLO, DHEA, pharmaceutically acceptable salts thereof, derivatives thereof, or combinations thereof. In some embodiments, the effective amount is sufficient to raise the level of PG, ALLO, DHEA, derivatives thereof, or combinations thereof in a source selected from the group consisting of cerebrospinal fluid, serum, plasma, blood, saliva, skin, muscle, olfactory tissue, lacrimal fluid, synovial fluid, nail tissue, hair, feces, urine, in the subject by at least 1.5-fold within 8 weeks from a level in the source in the subject prior to the administering step. In some embodiments, the effective amount comprises a daily dose of at least 0.005 mg per day. In some embodiments, the effective dose comprises a dose ranging from about 0.005 mg to about 2000 mg of PG, ALLO, or DHEA, or an equivalent molar amount of the pharmaceutically acceptable salt thereof, the derivative thereof, or the combinations thereof. In some embodiments, the effective amount is sufficient to improve a cognitive function in the subject. In some embodiments, the neuroactive steroid composition comprises an effective amount of each of two or more of PG, ALLO, DHEA, derivatives thereof, or combinations thereof.

For additional guidance regarding formulation and dose, see U.S. Pat. Nos. 5,326,902; 5,234,933; PCT International Publication No. WO 93/25521; Berkow et al., 1997; Goodman et al., 1996; Ebadi, 1998; Katzung, 2001; Remington et al., 1975; Speight et al., 1997; Duch et al., 1998.

IV.C. Routes of Administration

The presently disclosed neuroactive steroid compositions can be administered to a subject in any form and/or by any route of administration. In some embodiments, the formulation is a sustained release formulation, a controlled release formulation, or a formulation designed for both sustained and controlled release. As used herein, the term “sustained release” refers to release of an active agent such that an approximately constant amount of an active agent becomes available to the subject over time. The phrase “controlled release” is broader, referring to release of an active agent over time that might or might not be at a constant level. Particularly, “controlled release” encompasses situations and formulations where the active ingredient is not necessarily released at a constant rate, but can include increasing release over time, decreasing release over time, and/or constant release with one or more periods of increased release, decreased release, or combinations thereof. Thus, while “sustained release” is a form of “controlled release”, the latter also includes delivery modalities that employ changes in the amount of an active agent (e.g., a neuroactive steroid composition) that are delivered at different times.

In some embodiments, the sustained release formulation, the controlled release formulation, or the combination thereof is selected from the group consisting of an oral formulation, a peroral formulation, a buccal formulation, an enteral formulation, a pulmonary formulation, a rectal formulation, a vaginal formulation, a nasal formulation, a lingual formulation, a sublingual formulation, an intravenous formulation, an intraarterial formulation, an intracardial formulation, an intramuscular formulation, an intraperitoneal formulation, a transdermal formulation, an intracranial formulation, an intracutaneous formulation, a subcutaneous formulation, an aerosolized formulation, an ocular formulation, an implantable formulation, a depot injection formulation, a transdermal formulation and combinations thereof. In some embodiments, the route of administration is selected from the group consisting of oral, peroral, buccal, enteral, pulmonary, rectal, vaginal, nasal, lingual, sublingual, intravenous, intraarterial, intracardial, intramuscular, intraperitoneal, transdermal, intracranial, intracutaneous, subcutaneous, ocular, via an implant, and via a depot injection. Where applicable, continuous infusion can enhance drug accumulation at a target site (see e.g., U.S. Pat. No. 6,180,082). See also U.S. Pat. Nos. 3,598,122; 5,016,652; 5,935,975; 6,106,856; 6,162,459; 6,495,605; and 6,582,724; and U.S. Patent Application Publication No. 20060188558 for transdermal formulations and methods of delivery of compositions.

The particular mode of drug administration used in accordance with the methods of the presently disclosed subject matter depends on various factors, including but not limited to the vector and/or drug carrier employed, the severity of the condition to be treated, and mechanisms for metabolism or removal of the drug following administration.

### EXAMPLES

The following Examples provide illustrative embodiments. Certain aspects of the following Examples are disclosed in terms of techniques and procedures found or contemplated by the present inventors to work well in the practice of the embodiments. In light of the present disclosure and the general level of skill in the art, those of skill will appreciate that the following Examples are intended to be exemplary only and that numerous changes, modifications, and alterations can be employed without departing from the scope of the presently claimed subject matter.

### Materials and Methods Employed in the Examples

Study Design and Subjects. Disclosed herein is a placebo-controlled, double-blind, parallel group, randomized trial of adjunctive pregnenolone for the treatment of cognitive and negative symptoms in patients with schizophrenia or schizoaffective disorder. Following a single-blind 2-week placebo lead-in phase (all patients), subjects were randomized to 8 weeks of treatment with adjunctive pregnenolone or placebo. Patients received a total of 6 study visits, which took place every two weeks. In addition, subjects received staggered telephone check-in calls to assess potential side effects every two weeks (i.e., during alternate weeks that a study visit did not take place). The trial was conducted at a single site, the Durham Veterans Affairs Medical Center in Durham, N.C. The protocol was approved by the local institutional review board and conducted under FDA Investigational New Drug (IND) #71,768. The ClinicalTrials.gov number for this study is NCT00560937.

Subjects were outpatients between the ages of 18 and 65 with schizophrenia or schizoaffective disorder diagnosed by DSM-IV/DSM-IV-TR criteria utilizing the Structured Clinical Interview (First et al., 1996) and medical records. All subjects were between the ages of 18-65 and provided informed consent. Duration of illness was greater than one year for all enrolled patients. To be eligible for the trial, patients had to be taking a second-generation antipsychotic (aripiprazole, olanzapine, quetiapine, or risperidone) for at least 8 weeks, with no changes in antipsychotic dosing in the preceding 4 weeks. Patients receiving first-generation antipsychotics were excluded. Concomitant psychiatric medications were permissible (antidepressants, mood stabilizers, anticholinergics, other), provided that patients were receiving stable doses of all of these medications in the 4 weeks preceding the trial and throughout the entire duration of the study. Any change in psychiatric medications at any point during the study rendered a patient ineligible to continue participation. Exclusionary criteria were unstable medical or neurological illness, alcohol or other substance dependence within the last month (other than nicotine), use of oral contraceptives or other hormonal supplementation such as estrogen (although early studies suggested no effects on menstrual cycle, alterations in downstream pregnenolone metabolites such as estradiol could theoretically impact the efficacy or oral contraceptives or estrogen replacement), active expression of suicidal or homicidal ideation, pregnancy or breast-feeding, and known allergy to study medication.

During the screening visit (Visit 1), patients received a physical exam, vital signs, and electrocardiogram (EKG), as well as baseline laboratory measures consisting of serum electrolytes, glucose, creatinine, blood urea nitrogen (chemistry 7 panel), liver function tests, complete blood count (CBC), urinalysis, urine toxicology test, thyroid stimulating hormone (TSH), and prolactin, and serum pregnancy test if female. These tests were also repeated at the completion of the study (Visit 6). In addition, patients received an interim EKG 4 weeks post-randomization, as well as a serum pregnancy test if female (Visit 4). Patients also received a chemistry 7 panel, CBC, and liver function tests at Visits 2, 3, 4, and 5 (i.e., at all study visits). Psychiatric symptoms were assessed at baseline using the Scale for the Assessment of Negative Symptoms (Andreasen, 1983), the Positive and Negative Symptom Scale (PANSS; Kay et al., 1987), the Calgary Depression Rating Scale (CDRS; Addington et al., 1990), the Clinical Global Impressions Improvement (CGI-I) and Severity (CGI-S) scales, the Barnes Akathisia Scale (BAS; Barnes, 1989), the Simpson Angus Scale for Extrapyramidal Symptoms (SAS; Simpson & Angus, 1970), the Quality of Life Scale (QOL; Heinrichs et al., 1984), and the Hillside Side Effects Scale. One research nurse with five years of experience in the conduction of schizophrenia clinical trials completed both psychiatric and cognitive assessments throughout the study, and was trained to one point of divergence on each PANSS item.

Cognitive symptoms were assessed by the Brief Assessment of Cognition in Schizophrenia (BAGS; Keefe et al., 2004; Keefe et al., 2008), and the MATRICS Consensus Cognitive Battery (Nuechterlein & Green, 2006; Nuechterlein et al., 2008). It is believed that no clinical trial to date has reported both of these cognitive assessments within the same study. The BAGS and MATRICS batteries were administered two weeks following the screening visit (following the completion of the two-week placebo lead-in phase at Visit 2), and at two 4-week intervals thereafter (at midpoint following randomization at Visit 4 and at the final visit (Visit 6).

The BAGS cognitive battery utilizes the following assessments in the respective targeted domains (Keefe et al., 2004; Keefe et al., 2008): list learning (verbal memory), digit sequencing task (working memory), token motor task (motor speed), verbal fluency (processing speed), symbol coding (attention and processing speed), and the Tower of London test (executive functions/reasoning and problem solving). The MATRICS cognitive battery utilizes the following assessments: Trail Making Test, Part A and the symbol coding subtest of the BAGS (both assess speed of processing), the Hopkins Verbal Learning Test-Revised, immediate recall (three learning trials only) [verbal learning], the Wechsler Memory Scale, 3rd ed., spatial span subtest (working memory, nonverbal), the Letter-Number Span test (working memory, verbal), the Neuropsychological Assessment Battery, mazes subtest (reasoning and problem solving), the Brief Visuospatial Memory Test-Revised (visual learning), the Category Fluency Test (animal naming) [speed of processing], the Mayer-Salovey-Caruso Emotional Intelligence Test, managing emotions branch (social cognition), and the Continuous Performance Test, Identical Pairs version (attention/vigilance) Of note, the symbol coding subtest of the BAGS was administered only once per visit at Visits 2, 4, and 6, although it is a component of both the BAGS and MATRICS cognitive batteries, in order to avoid potentially confounding practice effects.

Study drug (pregnenolone (PREG); 50 mg) and matching placebo identical in appearance was obtained from Douglas Laboratories (Pittsburgh, Pa.), which provided certificates of analysis for pregnenolone 50 mg tablets and matching placebo. Study drug was dispensed every 2 weeks at each study visit at fixed escalating doses as follows: Visit 1: Two-week single-blind placebo lead-in phase, one placebo tablet twice each day (all pts), Visit 2: Randomization to PREG 50 mg twice each day (100 mg/day total) or placebo for 2 wks, Visit 3: PREG 150 mg twice each day (300 mg/day total) or placebo for 2 weeks, Visit 4: PREG 250 mg twice each day (500 mg/day total) or placebo for 2 weeks, Visit 5: PREG 250 mg twice each day (500 mg/day total) or placebo continued for 2 weeks. Patients thus received PREG 500 mg per day in divided doses during the last 4 weeks of the study. Patients also received the Hillside Adverse Events Scale at Visits 2-6, and during staggered telephone check-in calls every two weeks, beginning one week into the single-blind placebo lead-in phase.

Neurosteroid Analyses. Pregnenolone and allopregnanolone levels in serum were determined by a highly sensitive and specific gas chromatography/mass spectrometry (GC/MS) method in the negative ion chemical ionization mode, as previously described (Marx et al., 2006d; Marx et al., 2006e). One mL of serum was extracted three times in ethyl acetate prior to high performance liquid chromatography (HPLC) purification utilizing tetrahydrofuran, ethanol, and hexane in the mobile phase. All samples were injected in duplicate. The mean coefficients of variation for pregnenolone and allopregnanolone were 1.9% and 4.9%, respectively. The limit of detection with this method was 2 picograms for both pregnenolone and allopregnanolone. Pregnenolone sulfate levels in serum were determined by radioimmunoassay (RIA) as previously described (Porcu et al., 2008), with modifications. All other serum steroid levels were determined utilizing commercially available radioimmunoassay (RIA) kits according to manufacturer directions (progesterone, cortisol, testosterone, free testosterone, and DHEAS: Diagnostic System Laboratories, Webster, Tex.). DHEA serum levels were determined utilizing a kit from ICN Pharmaceuticals (Costa Mesa, Calif.).

Statistical Analysis. Primary endpoints were changes from baseline at Visit 2 following a two-week placebo lead-in period (all patients) to final assessment at Visit 6 in cognitive symptoms (as assessed by BAGS and MATRICS composite scores) and negative symptoms (as assessed by SANS total scores). If the final Visit 6 assessment was missing for a subject (8 weeks of treatment with pregnenolone or placebo post-randomization), Visit 4 was carried forward for BAGS, MATRICS, SANS, and other psychiatric assessments (4 weeks of treatment with pregnenolone or placebo post-randomization). Student's t-tests were conducted on the change scores between treatment groups in this proof-of-concept study (two-tailed alpha p=0.05 for each statistical test). Spearman correlation coefficients were determined to assess the relationship between increases in serum pregnenolone levels and cognitive and psychiatric improvements, as well as the relationship between baseline neurosteroid levels and changes in these outcome measures following treatment with pregnenolone. Paired t-tests were utilized to assess pregnenolone and pregnenolone metabolite level changes in serum post-treatment.

### Example 1

### Subject Characterization

Of 28 subjects who received a screening visit, 21 met entry criteria and were randomized at Visit 2 to 8 weeks of treatment with adjunctive pregnenolone or placebo, following a 2-week single-blind placebo lead-in phase for all patients. The number of subjects who completed 4 or more weeks of treatment with pregnenolone or placebo post-randomization was 18 (86%), with 17 patients completing the entire 8-week study post-randomization (81%). One patient randomized to the placebo group completed only 4 weeks of the study secondary to requiring an increase in antipsychotic dose, thus excluding this subject from continued participation (Visit 4 assessments were thus carried forward for the statistical analyses). Baseline demographic and clinical characteristics are presented in Table 1. The mean age of subjects was 49.43 years (±12.19 SD) for the placebo group and 52.68 (±6.31 SD) for the pregnenolone group. The study sample contained only one female subject, reflecting the approximate demographic of the population with psychotic disorders treated at the Durham Veterans Affairs Medical Center.

TABLE 1
Baseline Demographic and Clinical Characteristics

| | Placebo | PG |
|---|---|---|
| | (n = 9) | (n = 9) |
| Male/Female | 9/0 | 8/1 |
| Age (yrs) | 49.4 ± 12.2 | 52.7 ± 6.3 |
| Schizophrenia | 5 | 6 |
| Schizoaffective Disorder | 4 | 3 |
| Caucasian | 3 | 3 |
| African American | 5 | 6 |
| Other | 1 | 0 |
| WRAT | 100.1 ± 15.3 | 101.1 ± 10.9 |

| | Placebo | | Pregnenolone | |
|---|---|---|---|---|
| Antipsychotic | Number of | Mean Dose | Number of | Mean Dose |
| (mg/ml) | Subjects | (±SD) | Subjects | (±SD) |
| Aripiprazole | 1 | 15 ± 0 | 2 | 30 ± 21 |
| Olanzapine | 5 | 18 ± 2.7 | 3 | 13.3 ± 7.6 |
| Quetiapine | 1 | 300 ± 0 | 2 | 400 ± 283 |
| Risperidone | 2 | 5 ± 1.4 | 2 | 6 ± 0 |

Of 18 randomized subjects completing at least 4 weeks of the study, 3 were treated with aripiprazole, 8 with olanzapine, 3 with quetiapine, and 4 with risperidone (see Table 1). Three of the 21 patients who were randomized did not complete at least four weeks of treatment with pregnenolone or placebo for the following reasons:
1) One patient began truck-driving school shortly after randomization at Visit 2 and his new schedule and location could not accommodate study visits;
2) One patient arrived intoxicated at Visit 4 following heavy alcohol and cocaine use that morning, and had taken his oral hypoglycemic without breakfast. He subsequently sustained an episode of hypoglycemia requiring medical attention that was believed to be unrelated to study drug, per internal medicine physician evaluation on the day of the event; and
3) One patient received an increase in antipsychotic prescribed by the subject's regular psychiatrist between Visit 2 and Visit 3, hence making the patient ineligible to continue in the study.

Missing data were addressed in the following manner. For cognitive assessments, any missing subscale was imputed as directed by BAGS and MATRICS guidelines, and these imputed values are included in the cognitive subscale tables for the BAGS and MATRICS described below. There were relatively few missing cognitive data for either the BAGS or MATRICS instruments and testing sessions were generally very well-tolerated. For the BAGS, 1% of the total number of administered subscales was missing and required imputation (one symbol coding test and two token motor tests). For the MATRICS, 1.5% of the total number of administered subscales was missing (two continuous performance tests [CPT] and five social cognition tests [MSCEIT]). One patient received BAGS and MATRICS cognitive batteries and CGI assessments at baseline, but could not complete several other rating scales (PANSS, SANS, Calgary Depression Rating Scale) secondary to a scheduling conflict. Since sleep deprivation for 20-25 hours significantly impacts cognitive testing performance in a manner comparable to a blood alcohol level of 0.10% (Dawson & Reid, 1997; Lamond & Dawson, 1999), cognitive data for one patient who reported sleep deprivation for the preceding 24 hours at the final study visit could not be utilized; this patient's interim cognitive assessments from Visit 4 were thus carried forward for the statistical analyses. Other missing assessments were addressed via casewise deletion (3 WRAT and 1 Calgary Depression Rating Scale), as were three outlying steroid levels greater than 2.4 standard deviations above the mean that were omitted from analyses as per statistical consultation. Of the serum estradiol and free testosterone levels determined at baseline and at the final study visit in both the pregnenolone and placebo groups (n=35; one randomized patient did not reach the final study visit as described above), six estradiol and two free testosterone levels were below the limits of detection of the RIA kits utilized in this investigation (8 pg/mL and 0.15 pg/mL, respectively).

### Example 2

### Pregnenolone Significantly Reduces SANS Scores Compared to Placebo

Baseline SANS scores were similar in both the placebo and pregnenolone groups (50.75±12.21 SD and 47.56±12.09 SD, respectively). Patients randomized to pregnenolone completing at least 4 weeks of treatment post-randomization demonstrated significantly improved SANS scores (mean change 10.38±10.18 SD) compared to patients receiving placebo (mean change 2.33±4.42 SD), unpaired Student's t-test of SANS change from baseline t=2.16, df=15, p=0.048, see Table 2. Pregnenolone thus outperformed placebo by approximately 8 points on the SANS assessment, a difference that might be clinically relevant. SANS subscales are also provided in Table 2, although this information should be interpreted with caution given the small number of subjects in this study. This table does, however, provide data for future hypothesistesting with regard to domains of the SANS that might be responsive to a pregnenolone intervention. In the subscale analyses, uncorrected for multiple comparisons in this exploratory investigation, patients randomized to pregnenolone demonstrated significantly greater improvements in the “affect” subscale of the SANS (t=2.33, df=15, p=0.035), and tended to demonstrate greater improvements in the “alogia” SANS subscale (t=1.83, df=15, p=0.087).

TABLE 2
SANS Subscales and Total Scores
SANS Subscales*

| | | | | | |
|---|---|---|---|---|---|
| | | Avolition/ | Anhedonia/ | | |
| Affect | Alogia | apathy | asociality | Attention | SANS Total |

Week 0

| | | | | | | |
|---|---|---|---|---|---|---|
| Placebo | 12.8 ± 4.0 | 8.8 ± 3.8 | 8.4 ± 2.4 | 11.6 ± 5.6 | 6.0 ± 1.0 | 47.6 ± 12.1 |
| (n = 8) | | | | | | |
| PG | 13.4 ± 5.0 | 8.8 ± 3.6 | 8.3 ± 2.5 | 12.0 ± 4.5 | 5.6 ± 1.1 | 50.8 ± 12.2 |
| (n = 8) | | | | | | |

Week 4

| | | | | | | |
|---|---|---|---|---|---|---|
| Placebo | 13.4 ± 4.5 | 8.4 ± 3.7 | 8.6 ± 2.5 | 11.1 ± 5.5 | 5.8 ± 1.2 | 47.3 ± 11.7 |
| (n = 8) | | | | | | |
| PG | 14.3 ± 3.2 | 7.8 ± 3.2 | 8.1 ± 1.0 | 10.4 ± 4.1 | 5.3 ± 1.0 | 45.8 ± 8.8 |
| (n = 8) | | | | | | |

Week 8

| | | | | | | |
|---|---|---|---|---|---|---|
| Placebo | 13.0 ± 4.4 | 8.4 ± 3.7 | 8.1 ± 2.7 | 10.8 ± 5.1 | 5.9 ± 1.5 | 45.2 ± 12.0 |
| (n = 8) | | | | | | |
| PG | 11.9 ± 0.4 | 6.9 ± 2.1 | 8.0 ± 0.5 | 9.6 ± 3.5 | 4.0 ± 0 | 40.4 ± 3.5 |
| (n = 8) | | | | | | |

Change from Week 0 to Week 8

| | | | | | | |
|---|---|---|---|---|---|---|
| Placebo | 0.2 ± 1.0 | −0.3 ± 0.3 | −0.3 ± 0.4 | −0.8 ± 0.5 | −1.1 ± 0.4 | −2.3 ± 4.4 |
| PG | −4.3 ± 1.7 | −1.9 ± 0.8 | −0.3 ± 0.3 | −2.4 ± 1.3 | −1.6 ± 0.4 | −10.4 ± 10.2 |
| Difference | −4.5 ± 1.9 | −1.5 ± 0.8 | 0.1 ± 0.6 | −1.6 ± 1.3 | −0.5 ± 0.5 | −8.0 ± 3.7 |

Statistics

| | | | | | | |
|---|---|---|---|---|---|---|
| t | 2.33 | 11.83 | 0.15 | 1.22 | 1.00 | 2.16 |
| df | 15 | 15 | 15 | 15 | 15 | 15 |
| p | 0.035 | 0.087 | 0.88 | 0.24 | 0.33 | 0.048 |

*Data in each column is provided as mean ± SD (Unpaired Student's t-tests conducted on change scores at Week 8 compared with Week 0).

### Example 3

### BAGS and MATRICS Assessments

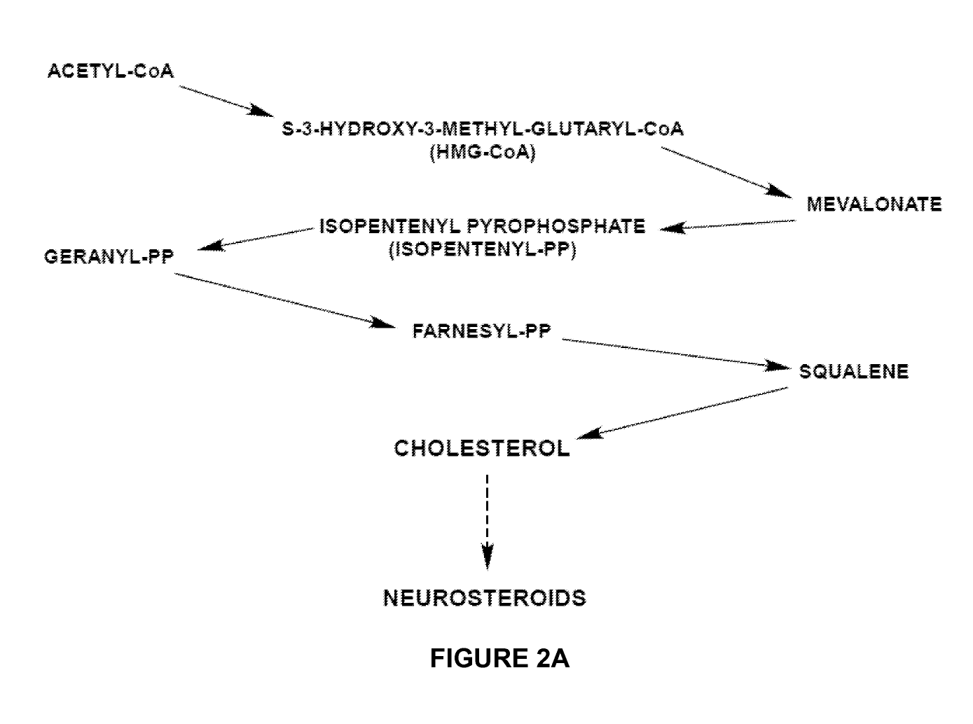

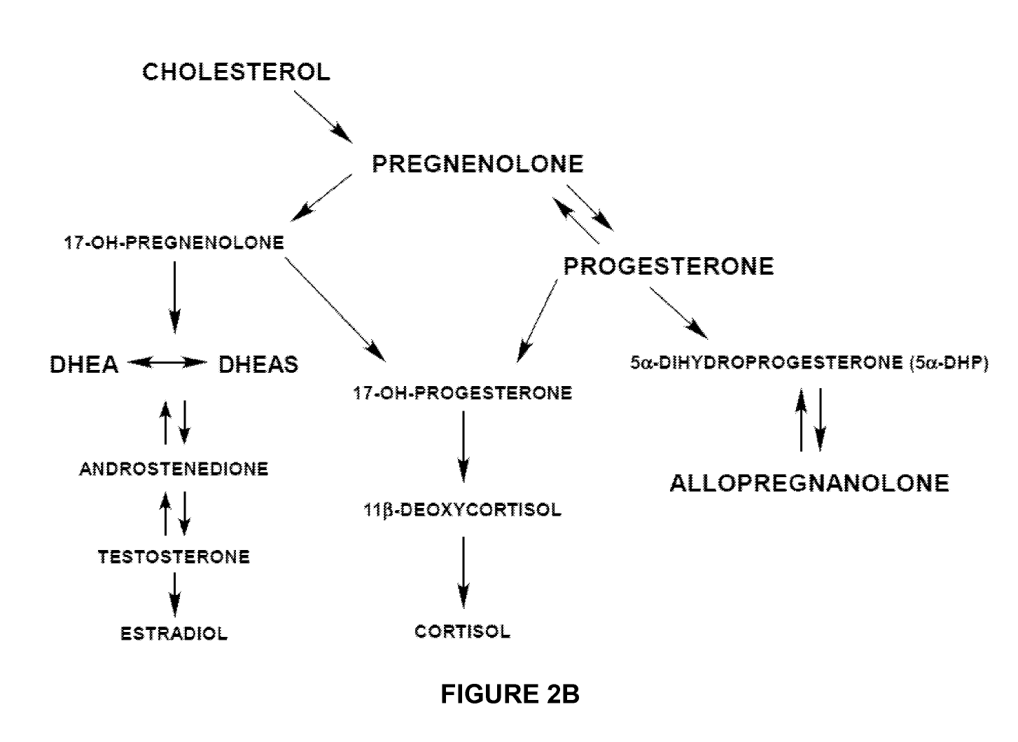

In the group randomized to pregnenolone, increases in serum levels of this neurosteroid predicted improvements in BAGS composite scores (Spearman r=0.81, n=8, p=0.022), suggesting that peripheral pregnenolone levels could potentially have biomarker utility for the assessment of clinical response (see FIG. 1A). Serum pregnenolone levels also tended to predict improvements in MATRICS composite scores (Spearman r=0.62, n=8, p=0.115; see FIG. 2B). Spearman correlation coefficients were determined (rather than Pearson) in order to reduce the influence of outliers (for example, see FIGS. 1B and 1C). If a Pearson correlation coefficient is utilized for the preceding analysis, however, the relationship between changes in serum pregnenolone levels post-treatment and improvements in MATRICS composite scores approaches statistical significance (Pearson r=0.70, n=8, p=0.053).

Serum increases in the GABAergic neurosteroid allopregnanolone also predicted improvements in BAGS composite scores (see FIG. 1C; Spearman r=0.74, n=8, p=0.046). Serum increases in pregnenolone sulfate levels did not predict improvements in the BAGS or MATRICS composite scores (p>0.05 for both assessments). However, baseline pregnenolone sulfate levels were inversely correlated with improvements in MATRICS composite scores (r=−0.83, n=8, p=0.015), suggesting that patients with lower pregnenolone sulfate levels at baseline experienced greater cognitive improvements as assessed by the MATRICS following treatment with adjunctive pregnenolone (see FIG. 1D). Similarly, baseline pregnenolone (r=−0.76, n=8, p=0.037) and allopregnanolone (r=−0.83, n=8, p=0.015) levels were also inversely associated with improvements in MATRICS composite scores, and baseline pregnenolone levels tended to be inversely associated with improvements in BAGS composite scores (r=−0.71, n=8, p=0.058).

Mean improvements in BAGS composite scores in the pregnenolone group (z-score change 0.60±0.78 SD) were greater than those in the placebo group (z-score change 0.22±0.47 SD) by a z-score of approximately 0.4 and approached a medium effect size, although this finding did not achieve statistical significance (p=0.22, t=1.27, df=16; see Table 3). Individual BAGS subscales are also presented in Table 3 for descriptive purposes to inform future hypothesis-testing. BAGS subscales that might demonstrate the greatest improvements in the pregnenolone group compared to the placebo group are verbal memory and the Tower of London tests (both with z-score improvements 0.61 greater than placebo), followed by the token motor and digit sequencing tasks in which the pregnenolone group demonstrated z-score improvements of 0.42 and 0.25, respectively, greater than placebo (see Table 3). Mean improvements in MATRICS composite scores were the same post-treatment in the pregnenolone (mean t-score change 7.00±8.87 SD) and placebo groups (mean t-score change 7.00±4.95 SD), a finding that appears driven by improvements in the MSCEIT subscale in the placebo group, (see Table 4). MATRICS composite scores and BAGS composite scores administered at baseline and post-treatment were positively correlated (Spearman correlation coefficient 0.74, p<0.0001, n=36).

TABLE 3
BACS Subscales and Composite Scores*

| | | | | | | | Changes in Z−scores from Week 0 | | |
|---|---|---|---|---|---|---|---|---|---|
| | Week 0 Raw Scores | | Week 4 Raw Scores | | Week 8 Raw Scores | | to Week 8 | | |
| | Placebo | PG | Placebo | PG | Placebo | PG | Placebo | PG | Difference |
| BACS | | | | | | | | | |
| Subscales | | | | | | | | | |
| Verbal | 35.33 ± | 36.78 ± | 37.89 ± | 39.00 ± | 37.22 ± | 43.78 ± | 0.22 ± 0.71 | 0.83 ± | 0.61 ± 0.38 |
| Memory | 12.33 | 7.82 | 10.51 | 7.12 | 11.29 | 9.80 | | 0.88 | |
| Digit | 18.44 ± | 15.78 ± | 19.56 ± | 17.22 ± | 19.78 ± | 18.44 ± | 0.25 ± 0.61 | 0.50 ± | 0.25 ± 0.29 |
| Sequencing | 3.17 | 3.99 | 3.17 | 3.35 | 4.79 | 3.91 | | 0.63 | |
| Token Motor | 53.67 ± | 46.44 ± | 53.11 ± | 55.56 ± | 53.33 ± | 52.22 ± | −0.02 ± 1.04 | 0.40 ± | 0.42 ± 0.66 |
| | 16.99 | 17.54 | 14.18 | 18.02 | 11.31 | 14.12 | | 1.67 | |
| Verbal | 44.00 ± | 45.78 ± | 42.78 ± | 45.11 ± | 44.11 ± | 45.22 ± | 0.01 ± 0.60 | −0.05 ± | −0.06 ± 0.32 |
| Fluency | 12.43 | 12.56 | 9.50 | 9.97 | 11.61 | 7.87 | | 0.75 | |
| Symbol | 36.78 ± | 36.89 ± | 39.44 ± | 44.78 ± | 41.22 ± | 38.67 ± | 0.27 ± 0.55 | 0.11 ± | −0.16 ± 0.24 |
| Coding | 9.32 | 13.39 | 9.61 | 11.97 | 10.01 | 13.61 | | 0.46 | |
| Tower of | 14.78 ± | 13.22 ± | 15.11 ± | 15.22 ± | 15.56 ± | 16.22 ± | 0.21 ± 1.62 | 0.82± | 0.61 ± 0.80 |
| London | 7.89 | 6.26 | 6.68 | 6.06 | 6.15 | 4.94 | | 1.79 | |
| | | | | | | | Changes in Z−scores from Week 0 | | |
| | Week 0 Z−Scores | | Week 4 Z−Scores | | Week 8 Z−Scores | | to Week 8 | | |
| | Placebo | PG | Placebo | PG | Placebo | PG | Placebo | PG | Difference |
| BACS | −1.28 ± | −1.54 ± | −1.14 ± | −1.04 ± | −1.06 ± | −0.93 ± | 0.22 ± 0.47 | 0.60 ± | 0.38 ± 0.30 |
| Composite | 0.96 | 0.96 | 0.80 | 0.75 | 0.88 | 0.87 | | 0.78 | |

*Data in each column are provided as Mean ± SD; both placebo and PG groups included 9 individuals.

TABLE 4
MATRICS/MCCB Subscales and Composite Scores*

| | Week 0 Raw | | Week 4 Raw | | Week 8 Raw | | Changes in Domain T-scores from | | |
|---|---|---|---|---|---|---|---|---|---|
| | Scores | | Scores | | Scores | | Week 0 to Week 8 | | |
| | Placebo | PG | Placebo | PG | Placebo | PG | Placebo | PG | Difference |
| MATRICS/MCCB | | | | | | | | | |
| Subscales | | | | | | | | | |
| | | | | | | | | Speed of | |
| | | | | | | | | Processing | |
| Trail Making Test, Part A | 56.11 ± | 42.11 ± | 42.33 ± | 38.33 ± | 44.11 ± | 34.33/ | 4.67 ± | 5.44 ± | 0.78 ± |
| | 30.29 | 8.37 | 11.62 | 7.76 | 10.52 | −7.12 | 7.50 | 8.69 | 3.83 |
| BACS, symbol coding | 36.78 ± | 36.89 ± | 39.44 ± | 44.78 ± | 41.22 ± | 38.67 ± | | | |
| subtest | 9.32 | 13.39 | 9.61 | 11.97 | 10.01 | 13.61 | | | |
| Category fluency test, | 18.00 ± | 18.33 ± | 16.44 ± | 18.44 ± | 18.67 ± | 20.44 ± | | | |
| animal naming | 5.63 | 5.17 | 4.64 | 3.78 | 4.21 | 4.1 | | | |
| | | | | | | | | Verbal | |
| | | | | | | | | learning | |
| Hopkins Verbal Learning | 21.00 ± | 18.56 ± | 20.33 ± | 20.89 ± | 21.11 ± | 21.00 ± | 0.78 ± | 3.44 ± | 2.67 ± |
| Test | 6.42 | 6.39 | 6.14 | 5.33 | 7.49 | 5.24 | 5.07 | 2.92 | 1.95 |
| | | | | | | | | Working | |
| | | | | | | | | memory | |
| Wechsler Memory Scale, | 12.00 ± | 13.11 ± | 12.11 ± | 14.33 ± | 12.78 ± | 14.22 ± | 1.11 ± | 5.11 ± | 4.00 ± |
| 3^{rd} ed., spatial span | 3.67 | 2.93 | 3.41 | 3.35 | 2.54 | 3.15 | 8.15 | 6.83 | 3.54 |
| subtest | | | | | | | | | |
| Letter-Number Span test | 13.22 ± | 11.44 ± | 13.33 ± | 12.67 ± | 13.11 ± | 13.11 ± | | | |
| | 2.91 | 3.13 | 3.91 | 3.08 | 4.11 | 3.86 | | | |
| | | | | | | | | Reasoning | |
| | | | | | | | | and Problem | |
| | | | | | | | | Solving | |
| Neuropsychological | 11.11 ± | 13.56 ± | 13.33 ± | 15.67 ± | 15.00 ± | 17.33 ± | 6.22 ± | 6.22 ± | 0.00 ± |
| Assessment Battery, | 3.92 | 6.42 | 6.40 | 6.24 | 6.87 | 6.30 | 9.01 | 10.80 | 4.69 |
| mazes subtest | | | | | | | | | |
| | | | | | | | | Visual | |
| | | | | | | | | learning | |
| Brief Visuospatial Memory | 16.33 ± | 20.22 ± | 18.78 ± | 21.33 ± | 20.78 ± | 22.33 ± | 7.56 ± | 3.78 ± | −3.78 ± |
| Test-Rev. | 8.15 | 8.63 | 9.04 | 10.07 | 10.24 | 9.30 | 13.44 | 7.87 | 5.19 |
| | | | | | | | | Social | |
| | | | | | | | | cognition | |
| Mayer-Salovey-Caruso | 86.87 ± | 86.89 ± | 90.85 ± | 90.60 ± | 93.92 ± | 86.10 ± | 8.33 ± | −0.89 ± | −9.22 ± |
| Emotional Intelligence | 13.30 | 14.84 | 16.12 | 13.55 | 12.43 | 16.81 | 6.60 | 15.57 | 5.64 |
| Test, managing | | | | | | | | | |
| emotions branch | | | | | | | | | |
| | | | | | | | | Attention/ | |
| | | | | | | | | vigilance | |
| Continuous Performance | 1.96 ± | 1.42 ± | 2.10 ± | 1.37 ± | 2.01 ± | 1.91 ± | 0.67 ± | 7.56 ± | 6.89 ± |
| Test (CPT), Identical | 0.97 | 0.93 | 1.11 | 1.24 | 1.19 | 1.05 | 5.45 | 19.45 | 6.73 |
| Pairs version-Average d′ | | | | | | | | | |
| | Week 0 Raw | | Week 4 Raw | | Week 8 Raw | | Changes in Domain T-scores from | | |
| | Scores | | Scores | | Scores | | Week 0 to Week 8 | | |
| | Placebo | PG | Placebo | PG | Placebo | PG | Placebo | PG | Placebo |
| MATRICS/MCCB | 27.33 ± | 29.00 ± | 31.33 ± | 33.78 ± | 34.33 ± | 36.00 ± | 7.00 ± | 7.00 ± | 0.00 ± |
| Composite | 12.96 | 10.06 | 15.04 | 11.66 | 14.07 | 10.97 | 4.95 | 8.87 | 3.39 |

*Data in each column are provided as Mean ± SD; both placebo and PG groups included 9 individuals.

### Example 4

### Psychiatric Assessments

Patients randomized to pregnenolone demonstrate significantly improved final CGI-I scores (2.11±0.33 SD), compared to the group randomized to placebo (2.89±0.78 SD), unpaired Student's t-test p=0.015, t=2.75, df=16 [CGI-I of 2=much improved; CGI-I of 3=minimally improved]; see FIG. 2. CGI-S scores were similar in both the pregnenolone and placebo groups at baseline (4.00±0.50 and 4.00±0.00, respectively) and post-treatment (3.89±0.33 and 4.00±0.00, respectively). PANSS scores were decreased by 4.43±3.59 more points in the pregnenolone group compared to the placebo group, but this finding was not significant (p=0.24, t=1.23, df=15; see Table 5). Pregnenolone administration tended to decrease the positive symptom subscale of the PANSS (p=0.07, t=1.96, df=15), but not the negative symptom or general subscales (see Table 5). The Heinrich Carpenter Quality of Life Scale improved by 5.27±5.07 points in the group randomized to pregnenolone compared to the placebo group but this finding was not statistically significant (p=0.32, t=1.04, df=14; see Table 5). There were no differences between the Calgary Depression Rating Scale, Barnes Akathisia Scale, Simpson Angus Scale or AIMS in the pregnenolone group and placebo group post-treatment. See Table 5 for summary of clinical assessments.

TABLE 5
Psychiatric Rating Scales by Treatment Group*

| | Week 0 | | Week 4 | | Week 8 | | Changes from Week 0-8 | | | Statistics | | |
|---|---|---|---|---|---|---|---|---|---|---|---|---|
| | Placebo | PG | Placebo | PG | Placebo | PG | Placebo | PG | Difference | t | df | p-value |
| PANSS total | 72.9 ± | 72.5 ± | 70.4 ± | 67.9 ± | 67.4 ± | 63.6 ± | −5.44 ± | −9.9 ± | −4.4 ± 3.6 | 1.23 | 15 | 0.24 |
| | 6.6 | 14.7 | 6.3 | 10.5 | 6.0 | 6.2 | 5.3 | 9.2 | | | | |
| | | | | | | | | (8) | | | | |
| PANSS subscores | | | | | | | | | | | | |
| Positive | 13.9 ± | 13.6 ± | 13.2 ± | 12.8 ± | 12.9 ± | 11.9 ± | −1.0 ± | −2.6 ± | −1.6 ± 0.8 | 1.96 | 15 | 0.069 |
| symptoms | 3.0 | 4.9 | 2.1 | 3.8 | 3.8 | 4.0 | 1.6 | 1.9 | | | | |
| | | | | | | | | (8) | | | | |
| Negative | 24.1 ± | 23.4 ± | 23.7 ± | 23.2 ± | 22.9 ± | 20.6 ± | −1.22 ± | −2.8 ± | −1.5 ± 1.4 | 1.11 | 15 | 0.29 |
| symptoms | 3.7 | 3.9 | 3.0 | 4.5 | 2.4 | 2.0 | 2.7 | 3.0 | | | | |
| | | | | | | | | (8) | | | | |
| General | 34.9 ± | 35.5 ± | 33.6 ± | 34.3 ± | 31.7 ± | 31.2 ± | −3.22 ± | −4.5 ± | −1.3 ± 2.1 | 0.61 | 15 | 0.55 |
| | 3.2 | 6.7 | 3.0 | 4.8 | 2.8 | 2.8 | 3.8 | 4.9 | | | | |
| | | | | | | | | (8) | | | | |
| CGI-I | — | — | — | — | 2.9 ± 0.8 | 2.11 ± | — | — | — | 2.75 | 16 | 0.014 |
| | | | | | | 0.3 | | | | | | |
| CGI-S | 4.0 ± 0.0 | 4.0 ± | — | — | 4.0 ± 0.0 | 3.9 ± | 0.0 ± 0.0 | −0.1 ± | NA | — | — | — |
| | | 0.5 | | | | 0.3 | | 0.3 | | | | |
| Calgary | 2.9 ± 4.1 | 2.4 ± | — | — | 0.9 ± 1.7 | 0.9 ± | −2.0 ± | −1.6 ± | 0.4 ± 1.6 | 0.27 | 13 | 0.79 |
| Depression | | 3.4 | | | | 1.7 | 3.7 | 2.2 | | | | |
| Scale | | | | | | | (8) | (7) | | | | |
| Simpson-Angus | 0.6 ± 0.7 | 1.0 ± | 0.9 ± 1.2 | 1.3 ± | 0.7 ± 0.9 | 0.9 ± | 0.1 ± 0.6 | −0.1 ± | −0.2 ± 0.4 | 0.68 | 15 | 0.51 |
| Scale | | 1.7 | | 1.7 | | 1.4 | | 0.8 | | | | |
| | | | | | | | | (8) | | | | |
| Barnes Akathisia | 0.0 ± 0.0 | 0.4 ± | 0.3 ± 1.0 | 0.4 ± | 0.0 ± 0.0 | 0.0 ± | 0.0 ± 0.0 | −0.4 ± | NA | — | — | — |
| Scale | | 1.1 | | 1.1 | | 0.0 | | 1.1 | | | | |
| | | | | | | | | (8) | | | | |
| AIMS | 1.7 ± 2.1 | 1.6 ± | 1.0 ± 1.7 | 0.9 ± | 1.2 ± 1.8 | 0.6 ± | −0.4 ± | −1.0 ± | −0.6 ± 0.7 | 0.61 | 15 | 0.55 |
| | | 2.1 | | 1.3 | | 0.9 | 1.9 | 1.9 | | | | |
| | | | | | | | | (8) | | | | |
| Heinrich Carpenter | 38.3 ± | 47.5 ± | — | — | 41.0 ± | 55.4 ± | 2.7 ± 7.2 | 7.9 ± | 5.3 ± 5.1 | 1.04 | 14 | 0.32 |
| Quality of Life | 14.9 | 14.8 | | | 15.3 | 17.9 | (8) | 12.4 | | | | |
| Scale | | | | | | | | (8) | | | | |
| Heinrich Carpenter | | | | | | | | | | | | |
| Subscores | | | | | | | | | | | | |
| Interpersonal and | 11.9 ± | 17.9 ± | — | — | 13.7 ± | 23.3 ± | 1.8 ± 4.1 | 5.4 ± | 3.6 ± 3.2 | 1.13 | 14 | 0.28 |
| social work | 6.9 | 9.1 | | | 6.4 | 8.0 | (8) | 8.1 | | | | |
| | | | | | | | | (8) | | | | |
| Occupational role | 1.6 ± 4.4 | 1.9 ± | — | — | 1.8 ± 5.0 | 2.8 ± | 0.1 ± 0.4 | 0.9 ± | 0.8 ± 0.6 | 1.26 | 14 | 0.23 |
| function | | 5.3 | | | | 6.3 | (8) | 1.6 | | | | |
| | | | | | | | | (8) | | | | |
| Other residual | 24.8 ± | 27.8 ± | — | — | 25.5 ± | 29.4 ± | 0.8 ± 3.0 | 1.6 ± | 0.9 ± 2.2 | 0.41 | 14 | 0.7 |
| symptoms | 6.8 | 5.9 | | | 6.3 | 6.7 | (8) | 5.3 | | | | |
| | | | | | | | | (8) | | | | |

*Data in each column are provided as Mean ± SD.
Both placebo and PG groups included 9 individuals unless a different number of individuals is indicated in parentheses.
NA: no calculation secondary to no change in CGI-S and Barnes Akathisia Scale scores in the placebo group.
Unpaired Student's t-tests conducted on change in scores at Week 8 compared to Week 0 (except for CGI-I, unpaired Student's t-test conducted on Week 8 assessments).

### Example 5

PG Administration Results in Increases in Serum PG and Specific Downstream Metabolite Levels, Including ALLO

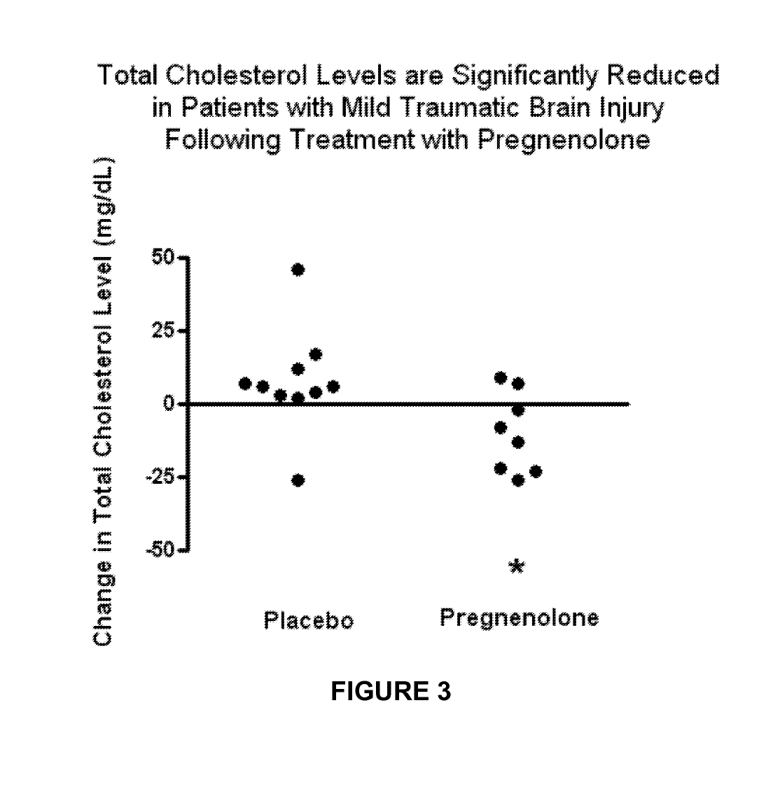

Paired t-tests were utilized to determine if PG administration alters the serum concentrations of a number of downstream metabolites, including the GABAergic neurosteroid ALLO and the NMDA receptor modulator PGS. Treatment with pregnenolone resulted in four-fold elevations in serum levels of PG (paired t-test p=0.017, t=3.11, df=7; see FIG. 3a), tripled serum PGS levels (paired t-test p<0.0001, t=10.44, df=8; see FIG. 3b), and increased the GABAergic neurosteroid ALLO five-fold (Paired t-test p=0.009, t=3.59, df=7; see FIG. 3c). PG administration also increased serum progesterone over four-fold and DHEAS levels by approximately 16% (see Table 6). Treatment with pregnenolone did not increase serum testosterone, free testosterone, cortisol, DHEA, estradiol, or androstenedione levels (see Table 6).

TABLE 6
Additional Steroid Levels at Baseline and Post-Treatment with PG

| | Week 0 | Week 8 | t | df | p-value |
|---|---|---|---|---|---|
| | Placebo (n = 8, unless specified) | | | | |
| PG (pg/ml) | 598.1 ± 329.6 | 514.0 ± 122.5 | 1.05 | 7 | 0.33 |
| PGS (ng/ml) | 52.2 ± 21.7 | 41.8 ± 24.3 | 1..98 | 7 | 0.09 |
| ALLO(pg/ml) | 105.6 ± 41.0 | 99.8 ± 51.4 | 0.43 | 7 | 0.68 |
| Cortisol (μg/100 ml) | 8.88 ± 5.13 | 8.84 ± 5.54 | 0.04 | 7 | 0.97 |
| Total testosterone | 399.31 ± 203.21 | 446.76 ± 140.36 | 1.03 | 7 | 0.34 |
| (ng/100 ml) | | | | | |
| Free testosterone | 7.96 ± 2.45 | 9.34 ± 2.95 | 1.49 | 7 | 0.18 |
| (pg/ml) | | | | | |
| DHEA (ng/ml) | 7.09 ± 3.11 | 8.22 ± 4.19 | 2.01 | 7 | 0.09 |
| DHEAS (μg/100 ml) | 151.58 ± 108.99 | 160.96 ± 114.38 | 0.75 | 7 | 0.48 |
| Progesterone (ng/ml) | 0.54 ± 0.24 | 0.53 ± 0.23 | 0.39 | 7 | 0.71 |
| Estradiol (pg/ml) | 24.16 ± 8.42 | 25.66 ± 10.18 | 0.48 | 5 | 0.65 |
| | (n = 6) | (n = 6) | | | |
| Androstenedione | 3.71 ± 2.28 | 3.32 ± 1.45 | 0.93 | 7 | 0.38 |
| (ng/ml) | | | | | |
| | Pregnenolone (n = 8, unless specified) | | | | |
| PG (pg/ml) | 499.9 ± 313.6 | 2096.0 ± 999.4 | 4016 | 7 | 0.0042 |
| | (n = 8) | (n = 8) | | | |
| PGS (ng/ml) | 33.4 ± 18.3 | 94.4 ± 14.3 | 10.44 | 8 | <0.0001 |
| ALLO (pg/ml) | 110.7 ± 78.9 | 823.1 ± 528.9 | | | |
| | (n = 8) | (n = 8) | | | |
| Cortisol (μg/100 ml) | 8.08 ± 2.80 | 7.96 ± 3.85 | 0.13 | 8 | 0.90 |
| Total testosterone | 302.83 ± 158.53 | 303.08 ± 156.05 | 0.01 | 8 | 0.99 |
| (ng/100 ml) | | | | | |
| Free testosterone | 7.96 ± 1.07 | 7.31 ± 2.04 | 0.79 | 7 | 0.46 |
| (pg/ml) | (n = 8) | (n = 8) | | | |
| DHEA (ng/ml) | 7.85 ± 5.94 | 6.33 ± 3.26 | 1.48 | 8 | 0.18 |
| DHEAS (μg/100 ml) | 124.72 ± 86.52 | 144.87 ± 97.30 | 3.61 | 8 | 0.007 |
| Progesterone (ng/ml) | 0.38 ± 0.15 | 2.17 ± 0.64 | 7.47 | 7 | 0.0001 |
| | (n = 8) | (n = 8) | | | |
| Estradiol (pg/ml) | 24.64 ± 6.21 | 25.42 ± 4.73 | 0.32 | 6 | 0.76 |
| | (n = 7) | (n = 7) | | | |
| Androstenedione | 2.93 ± 1.30 | 3.01 ± 4.66 | 0.22 | 8 | 0.83 |
| (ng/ml) | | | | | |

### Example 6

### Side Effect Profiles in Patients Randomized to Preqnenolone and Placebo

While there were no significant changes in most laboratory parameters, including glucose, triglycerides, HDL, TSH, and prolactin post-treatment compared to baseline in the pregnenolone group (see Table 7), cholesterol levels were significantly decreased following treatment with pregnenolone compared to baseline (paired t-test t=3.53, df=8, p=0.008). It is noted that CGI-S scores were similar in both the pregnenolone and placebo groups at baseline (4.00±0.50 and 4.00±0.00, respectively) and post-treatment (3.89±0.33 and 4.00±0.00, respectively). PANSS scores were decreased by 4.43±3.59 more points in the pregnenolone group compared to the placebo group, but not all blood draws were conducted in a fasting state. Serum LDL levels were also significantly reduced in the pregnenolone group post-treatment (paired t-test t=5.16, df=6, p=0.002). There was no change in EKG QTc interval following treatment with pregnenolone (see Table 7). Pregnenolone was very well-tolerated. The only side effects present in the pregnenolone group to a greater degree than the placebo group were two reports of mild restlessness, one report of mild muscle pain/stiffness, and one report of mild cold extremities (see Table 8).

TABLE 7
Serum Analytes and EKG QTc Interval at Screening
and Final Visit in Patients Randomized to PG

| | Pregnenolone (n = 8, unless specified) | | | | |
|---|---|---|---|---|---|
| | Screening | Week 8 | t | df | p-value |
| Glucose | 117.9 ± 36.2 | 119.0 ± 33.5 | 0.15 | 8 | 0.88 |
| (mg/100 ml) | | | | | |
| Cholesterol | 185.8 ± 43.3 | 166.6 ± 36.2 | 3.53 | 8 | 0.008 |
| (mg/100 ml) | | | | | |
| HDL (mg/100 ml) | 39.3 ± 16.5 | 37.0 ± 17.4 | 1.18 | 7 | 0.28 |
| | (n = 8) | (n = 8) | | | |
| LDL (mg/100 ml) | 119.1 ± 26.0 | 90.9 ± 29.4 | 5.16 | 6 | 0.002 |
| | (n = 7) | (n = 7) | | | |
| Triglycerides | 194.3 ± 87.8 | 237.6 ± 119.5 | 1.79 | 8 | 0.11 |
| (mg/100 ml) | | | | | |
| TSH (μIU/ml) | 1.45 ± 0.64 | 1.26 ± 0.72 | 0.84 | 8 | 0.42 |
| Prolactin (ng/ml) | 7.69 ± 5.91 | 7.85 ± 6.47 | 0.34 | 8 | 0.74 |
| EKG QTc interval | 414.8 ± 19.2 | 406.8 ± 20.9 | 1.19 | 8 | 0.27 |
| (ms) | | | | | |

TABLE 8
Side Effect Profiles in Patents Receiving Placebo and PG

| | PLACEBO | PG |
|---|---|---|
| | (n = 9) | (n = 9) |
| SYMPTOM | N (%) | N (%) |
| Disorientation (to date, address, | 2 (22%)^{a} | 2 (22%)^{a} |
| mayor, or MD name) | | |
| Decreased Interest in Sex | 2 (22%)^{a} | 1 (11%)^{a} |
| Impaired Sexual Performance | 2 (22%)^{a} | 0 |
| Hypertension | 1 (11%)^{b} | 1 (11%)^{b} |
| Excitation/Agitation | 1 (11%)^{a} | 0 |
| Dry mouth | 1 (11%)^{a} | 1 (11%)^{a} |
| Malaise | 1 (11%)^{a} | 0 |
| Blurred Vision | 1 (11%)^{a} | 0 |
| Restlessness | 0 | 2 (22%)^{a} |
| Muscle Pain/Stiffness | 0 | 1 (11%)^{a} |
| Cold Extremities | 0 | 1 (11%)^{a} |
| Tremor | 0 | 0 |
| Headache | 0 | 0 |
| Insomnia | 0 | 0 |
| Drowsiness | 0 | 0 |
| Rigidity | 0 | 0 |
| Akathisia | 0 | 0 |
| Diarrhea | 0 | 0 |
| Nasal Congestion | 0 | 0 |
| Sweating | 0 | 0 |
| Joint Pain/Stiffness | 0 | 0 |
| Peripheral Edema | 0 | 0 |
| All Other Side Effects | 0 | 0 |

Values are change from baseline pre-randomization (Hillside AE Form).
Key to superscripts:
^{a}mild in all subjects;
^{b}mild (<145/90).

### Example 7

### Subjects with Mild TBI Treated with PG have a Statistically Significant Reduction in Total Cholesterol

Eight (8) subjects with mild traumatic brain injury (TBI) were administered pregnenolone for 8 weeks in a fixed escalating dosing schedule as follows: pregnenolone 50 mg twice a day orally for 2 weeks, followed by pregnenolone 150 mg twice a day orally for 2 weeks, followed by pregnenolone 250 mg twice a day orally for 4 weeks). Ten (10) additional subjects with mild TBI were given a placebo. The results are presented in FIG. 3.

As shown in FIG. 3, subjects with mild TBI that were treated with PG experienced a statistically significant reduction (t-test of change scores at baseline compared to study completion p=0.035) in total cholesterol (measured by percent change) as compared to subjects given a placebo. As such, PG itself, as well other neurosteroids including, but not limited to allopregnanolone (ALLO), dehydroepiandrosterone (DHEA), and progesterone (PROG), as well as precursors thereof, metabolites thereof, pharmaceutically acceptable salts thereof, derivatives thereof, and/or combinations thereof, can thus represent a novel therapeutic advance for the treatment of hypercholesterolemia and/or hyperlipidemia and/or symptoms associated with undesirable cholesterol biosynthesis.

### DISCUSSION OF THE EXAMPLES

Disclosed herein is the discovery that adjunctive pregnenolone significantly reduced negative symptoms in patients with schizophrenia or schizoaffective disorder as assessed by the SANS, and that increases in serum pregnenolone and allopregnanolone levels post-treatment predicted improvements in the BAGS cognitive assessment battery. Increases in serum pregnenolone also tended to predict improvements in MATRICS composite scores. Patients randomized to pregnenolone who demonstrated lower pregnenolone sulfate, pregnenolone, and allopregnanolone levels at baseline showed greater improvements in MATRICS composite scores. These data suggested that adjunctive pregnenolone could have utility for both negative and cognitive symptoms in schizophrenia. In addition, patients receiving adjunctive pregnenolone demonstrated significantly improved CGI-I scores. These findings, related results, and potential pregnenolone mechanisms of action are discussed below.

Adjunctive Pregnenolone Significantly Reduces Negative Symptoms. Treatment with adjunctive pregnenolone significantly reduced negative symptoms as assessed by SANS scores in patients with schizophrenia or schizoaffective disorder compared to placebo. A significant effect on negative symptoms following pregnenolone treatment was encouraging. In the present analyses, individual subscales of the SANS were also investigated. Results suggested that the greatest improvements in negative symptoms following adjunctive pregnenolone occurred in the “affective flattening or blunting” domain, with the next most pronounced improvements occurring in the “alogia” and “anhedoniaasociality” subscales.

The change in “affective flattening or blunting” was significantly greater in the pregnenolone group, uncorrected for multiple comparisons within the study. Since greater negative symptom improvement in the pregnenolone group appeared to occur among SANS subscales containing items that could overlap with depressive symptomatology (“affective flattening or blunting” and “anhedonia-asociality”), it is perhaps worth noting that patients with depressive symptoms demonstrate reductions in cerebrospinal fluid pregnenolone levels (George et al., 1994). It is therefore possible that supplementation with pregnenolone could be particularly efficacious for negative symptoms with potential affective dimensions. Supporting this possibility, the antidepressant fluoxetine elevates pregnenolone to physiologically relevant concentrations in rodent brain (Marx et al., 2006b; Serra et al., 2001), and induction of this neurosteroid could thus contribute to its therapeutic efficacy for depressive symptoms (Pinna et al., 2006). It is noted, however, that the cohort of patients in this investigation exhibited only a minimal degree of depressive symptomatology at baseline and demonstrated total mean Calgary Depression Rating Scale scores of less than 3 in each group (see Table 5).

A number of pregnenolone characteristics could play roles in the production of the aforementioned potential therapeutic effects. For example, pregnenolone enhances myelination (Koenig et al., 1995), increases neuritic outgrowth (Fontaine-Lenoir et al., 2006), and impacts microtubule polymerization and stability (Murakami et al., 2000; Fontaine-Lenoir et al., 2006; Hsu et al., 2006), actions that might be relevant to the pathophysiology of schizophrenia (Glantz & Lewis, 2000; Hakak et al., 2001; Benitez-King et al., 2004; Tkachev et al., 2007). Furthermore, it was determined previously that clozapine markedly elevates pregnenolone levels in rat hippocampus and serum to a greater degree than other second generation antipsychotics at doses producing comparable striatal D2 receptor occupancies, representing a candidate mechanism for its superior efficacy (Marx et al., 2006a). Recent evidence that clozapine enhances the binding of the translocator protein involved in the rate-limiting step leading to pregnenolone formation from cholesterol (formerly referred to as the peripheral-type benzodiazepine receptor, (Lacapere & Papadopoulos, 2003)) supports a role for this neurosteroid in clozapine mechanisms of action (Danovich et al., 2008). In addition, pregnenolone is present in human brain tissue at physiologically relevant concentrations in the nanomolar range that are greater than 10-fold higher than those frequently observed in serum or plasma (Marx et al., 2006c).

Pregnenolone is elevated in posterior cingulate and parietal cortex in patients with schizophrenia compared to control subjects, a finding that could represent compensatory changes and/or drug effects (Marx et al., 2006c), given prior evidence that pharmacological agents such as clozapine and olanzapine elevate neurosteroids (Barbaccia et al., 2001; Marx et al., 2003; Marx et al., 2006a; Marx et al., 2006b).

Based upon a strong rationale for possible NMDA receptor hypofunction in schizophrenia (Javitt, 2004; Millan, 2005; Coyle, 2006; Rujescu et al., 2006; Javitt, 2007), a number of prior investigations utilizing compounds with modulatory activities at NMDA receptors such as D-serine (Tsai et al., 1998; Tsai et al., 1999; Heresco-Levy et al., 2005), glycine (Heresco-Levy et al., 1996; Heresco-Levy et al., 1999; Javitt et al., 2001; Heresco-Levy et al., 2004; Buchanan et al., 2007), and D-cycloserine (Cascella et al., 1994; Goff et al., 1995; Goff et al., 1996; Rosse et al., 1996; van Berckel et al., 1996; Goff et al., 1999a; Goff et al., 1999b; Duncan et al., 2004; Buchanan et al., 2007) have been undertaken, with somewhat mixed findings. Because the presently disclosed data demonstrated that adjunctive pregnenolone administration significantly elevates serum levels of pregnenolone sulfate, a positive NMDA receptor modulator, it is possible that metabolism to this neurosteroid might contribute to its therapeutic efficacy for negative symptoms via this mechanism.

It is unknown, however, if pregnenolone-induced increases in serum pregnenolone sulfate also lead to elevated pregnenolone sulfate levels in human brain. A rodent investigation suggests this might be the case (Wang et al., 1997), but the accurate determination of pregnenolone sulfate levels in brain tissue has also been the subject of several recent inquiries addressing possible confounding methodological challenges in the quantification of this neurosteroid (Higashi et al., 2003a; Higashi et al., 2003b; Liu et al., 2003; Liere et al., 2004; Ebner et al., 2006; Schumacher et al., 2008). It is therefore possible that other mechanisms potentially contribute to pregnenolone effects on negative symptoms that are unrelated to the NMDA receptor modulatory actions of its sulfated metabolite.

Adjunctive Pregnenolone and Cognition in Schizophrenia. Serum increases in pregnenolone in the group randomized to this neurosteroid were significantly correlated with cognitive improvement following this intervention as determined by the BAGS assessment battery, and also tended to be correlated with improvements in MATRICS composite scores, findings that are encouraging with regard to the therapeutic potential of pregnenolone for cognitive symptoms in schizophrenia. These correlations also highlight the biomarker potential of serum pregnenolone levels in predicting therapeutic response. Along these lines, lower baseline pregnenolone sulfate, pregnenolone, and allopregnanolone levels were also associated with greater improvements in MATRICS composite scores. It is therefore possible that the determination of peripheral neurosteroid levels might have predictive utility for the identification of schizophrenia patients who would potentially benefit from a pregnenolone intervention and for the prediction of clinical efficacy.

While the present results indicated that increases in serum pregnenolone correlated with improved cognition, the question as to whether greater pregnenolone serum increases might be required for optimal cognitive therapeutic responses remains, and raises the issue of the most appropriate dosing strategy for this neurosteroid. It is possible, for example, that the dose and/or duration of pregnenolone 500 mg per day in the final 4 weeks of this 8-week study might be inadequate to achieve maximal cognitive improvement, or to facilitate cognitive enhancement in a subset of patients whose pregnenolone levels are not optimally elevated post-treatment. Extremely few pharmacokinetic data are currently available for this agent following acute administration (Morley et al., 1997; Roberts, 1995), and it is believed that data regarding peripheral pregnenolone levels following chronic administration in humans have not been published to date.

The experiments disclosed herein thus provide new data in this area. Patients randomized to pregnenolone in this investigation achieved or moderately exceeded serum levels of this neurosteroid within the higher end of typical physiological ranges observed in young adults, representing an approximate four-fold increase over baseline pregnenolone levels prior to randomization. It is possible, however, that pregnenolone dosing that achieves supra-physiological levels could be required to achieve optimal efficacy. Conversely, learning and memory data from behavioral rodent investigations following pregnenolone sulfate administration suggest that an inverted U-shaped curve could best model the relationship between pregnenolone sulfate dose and cognitive enhancement in animals (Akwa et al., 2001; Vallee et al., 2003). It is therefore possible that lower doses of pregnenolone than those utilized in this presently disclosed study could potentially be efficacious for cognitive enhancement.

In this study, the composite scores of the BAGS and MATRICS assessments were positively correlated (Spearman r=0.74, n=36, p<0.0001). Treatment with adjunctive pregnenolone non-significantly improved composite BAGS scores compared to placebo by a z-score of approximately 0.4, approaching a medium effect size if replicated in future studies adequately powered to detect this difference. There was no difference in mean composite MATRICS t-score changes in the pregnenolone group compared to the placebo group post-treatment, a finding that appears to be driven by improved performances on the MSCEIT (social cognition domain test) by the placebo group. It is not clear why the placebo group outperformed the pregnenolone group on this particular MATRICS measure, and this finding might have been impacted by a relatively larger number of missing MSCEIT assessments compared to other MATRICS subscales.

Adjunctive Pregnenolone and Other Psychiatric Outcome Measures. Patients randomized to pregnenolone demonstrated significantly greater improvements in CGI-I scores, demonstrating a mean post-treatment CGI-I score of 2.11 (2=much improved) compared to mean CGI-I scores of 2.89 in patients receiving placebo (3=minimally improved). A significant finding in this overall improvement measure (p<0.015) is potentially encouraging in a pilot investigation of this size. Another measure of general patient functioning, the Heinrich Carpenter Quality of Life Scale, was not significantly altered post-treatment in this initial trial, but an improvement of 5.27 points in the pregnenolone group compared to the placebo group was in the predicted direction (with possibly the greatest improvement in the “interpersonal and social work” subscale). Together these findings suggested a potential signal for overall improvement following pregnenolone administration.

With regard to PANSS total scores, patients randomized to pregnenolone demonstrated slightly greater improvements in this outcome measure compared to placebo (4.43 points), but the finding was not statistically significant. The negative symptom subscale of the PANSS was non-significantly improved to a modest degree by 1.53 points in the pregnenolone group compared to the placebo group. This result is not necessarily surprising, however, since the SANS demonstrates greater sensitivity for the assessment of this symptom domain compared to the PANSS negative symptom subscale, and hence the absence of a statistically significant effect could reflect limited power in this initial study of nine subjects per group.

Finally, treatment with pregnenolone tended to reduce the positive symptom subscale of the PANSS (p<0.07). As discussed above, there were no significant differences in improvements in depressive symptoms in the pregnenolone group compared to the placebo group, as assessed by the Calgary Depression Scale. This would perhaps be expected, since mean Calgary Depression Scale scores in both groups were already quite low at baseline in this relatively non-depressed cohort, and hence any further attenuation in depressive symptoms following treatment with pregnenolone might have been difficult to detect secondary to possible floor effects.

Pregnenolone Levels in Serum and Pregnenolone Metabolism Profiles: Candidate Biomarkers and Potential Relevance to Mechanisms of Action. Adjunctive pregnenolone elevates serum pregnenolone levels. Pregnenolone administration in this study resulted in approximately four-fold elevations in serum pregnenolone to physiologically relevant nanomolar levels. Preclinical data supported the possibility that reinstatement of pregnenolone levels to this extent could have significant beneficial effects. For example, pregnenolone enhances learning and memory in rodent models at even lower concentrations than the nanomolar serum concentrations achieved in the current investigation (Flood et al., 1992). Since pregnenolone is lipophilic and readily crosses the blood brain barrier, it is likely that brain concentrations of pregnenolone in humans are also elevated following pregnenolone administration, as suggested by animal studies (Wang et al., 1997). Further, it has previously been demonstrated that serum pregnenolone levels are closely correlated with hippocampal pregnenolone levels in rats (Marx et al., 2006a), and that cerebrospinal fluid (CSF) pregnenolone levels in humans are correlated with temporal cortex pregnenolone levels within the same subject cohort (Naylor et al., 2008). Serum pregnenolone (and possibly other neurosteroids) could thus serve as a proxy or surrogate marker for brain pregnenolone levels, potentially providing data that may be relevant to the prediction of clinical response.

Pregnenolone Metabolism to Other Neurosteroids.

Metabolism to Pregnenolone Sulfate. The data presented herein demonstrated that pregnenolone administration tripled serum levels of its sulfated derivative, pregnenolone sulfate. Pregnenolone sulfate levels attained in serum following pregnenolone administration were very consistent with doses required to achieve its positive effects on learning and memory in rodent models (Flood et al., 1992; Flood et al., 1995; Mathis et al., 1996; Meziane et al., 1996; Akwa et al., 2001). Furthermore, recent evidence suggests that pregnenolone sulfate positively modulates NMDA receptors at concentrations in the nanomolar range at binding sites distinct from a number of known NMDA receptor targets (Johansson et al., 2008). Pregnenolone sulfate in the nanomolar range also enhances long-term potentiation (Sliwinski et al., 2004) and influences GABA release (Mtchedlishvili & Kapur, 2003) in rodents.

In addition to these actions, pregnenolone sulfate might increase neurogenesis in rodent hippocampus (Mayo et al., 2005). As disclosed herein, patients with lower serum pregnenolone sulfate levels at baseline demonstrated greater cognitive improvements as assessed by the MATRICS. It is therefore possible that a subset of patients with schizophrenia could demonstrate relative pregnenolone sulfate deficits (as well as lower baseline pregnenolone and allopregnanolone levels) that are potentially restored to optimal levels with pregnenolone administration. Pregnenolone metabolism to pregnenolone sulfate could thus result in a number of additional mechanistic contributions for this neurosteroid and its effects on cognitive and negative symptoms, including the amelioration of hypothesized NMDA receptor hypofunction.

Metabolism to the GABAergic Neurosteroid Allopregnanolone. Pregnenolone administration resulted in five-fold elevations in serum allopregnanolone levels, and it is possible that pregnenolone metabolism to this downstream GABAergic neurosteroid metabolite might play a role in its therapeutic efficacy. Supporting this possibility, increases in allopregnanolone predicted cognitive improvement as assessed by BAGS composite scores. Given compelling evidence for a GABAergic deficit in patients with schizophrenia (Benes and Berretta, 2001; Lewis et al., 2003; Lewis et al., 2004; Guidotti et al., 2005; Lewis et al., 2005; Benes et al., 2007) and the fact that allopregnanolone potentiates GABAA receptor responses to a greater degree than benzodiazepines or barbiturates (Majewska et al., 1986; Morrow et al., 1987; Morrow et al., 1990), it is possible that pregnenolone metabolism to the GABAergic neurosteroid allopregnanolone contributes to its mechanism(s) of action.

Allopregnanolone elevations resulting from treatment with pregnenolone could also play a role in other physiological processes related to schizophrenia, since this GABAergic neurosteroid enhances neurogenesis (Wang et al., 2005) and demonstrates multiple pronounced neuroprotective properties (Lockhart et al., 2002; Griffin et al., 2004; Djebaili et al., 2005; Sayeed et al., 2006; Xilouri & Papazafiri, 2006; Mellon et al., 2008). In addition, it enhances myelination (Ghoumari et al., 2003) and possesses anticonvulsant actions (Kokate et al., 1994; Kokate et al., 1996).

Furthermore, allopregnanolone demonstrates multiple anti-inflammatory effects (He et al., 2004; VanLandingham et al., 2007) and decreases apoptosis (Charalampopoulos et al., 2004; Charalampopoulos et al., 2006; Xilouri & Papazafiri, 2006), actions that might be relevant to schizophrenia (Jarskog et al., 2005; Glantz et al., 2006; Dickerson et al., 2007; Knight et al., 2007; Lencz et al., 2007). Allopregnanolone also increases dopamine formation by tyrosine hydroxylase induction in the adrenal (Charalampopoulos et al., 2005). If this allopregnanolone effect also takes place in prefrontal cortex, it is possible that allopregnanolone elevations resulting from pregnenolone administration play a role in the attenuation of cognitive deficits in patients with schizophrenia, particularly those involving working memory and executive function.

Finally, allopregnanolone levels are decreased in postmortem brain tissue from patients with Alzheimer's disease (Marx et al., 2006e), and hence the restoration of allopregnanolone levels via precursor loading with pregnenolone may be a logical approach for a number of disorders in which the disruption of cognition is a salient characteristic.

Metabolism to Progesterone Intermediary. Abundant evidence demonstrates that progesterone plays an important role in a number of brain functions in addition to its well-characterized non-central nervous system effects. Progesterone is present in both male and female human brain in nanomolar concentrations (Lanthier & Patwardhan, 1986; Lacroix et al., 1987; Weill-Engerer et al., 2002). Progesterone administration results in pronounced neuroprotective effects in rodent models of traumatic brain injury (Roof et al., 1997; Djebaili et al., 2004; He et al., 2004; Djebaili et al., 2005; Jones et al., 2005; Robertson et al., 2006), and also positively impacts myelination processes (Koenig et al., 1995; Schumacher et al., 2000; Azcoitia et al., 2003; Ghoumari et al., 2003; Ibanez et al., 2003) and dendritic outgrowth during development (Sakamoto et al., 2001).

Extending these rodent investigations, a recent randomized controlled clinical trial reported that intravenous progesterone for three days reduced 30-day mortality rates by over 50% in acute moderate to severe traumatic brain injury (Wright et al., 2007), strongly supporting a neuroprotective role for this neurosteroid. This pivotal study has been described as the first successful clinical trial of a pharmacological agent for traumatic brain injury in 40 years, with no adverse events attributable to the intervention (Wright et al., 2007; Stein, 2008).

Furthermore, these promising clinical findings have recently been replicated, and also extended to demonstrate enduring therapeutic progesterone effects six months following traumatic brain injury (Xiao et al., 2008). As disclosed herein, pregnenolone administration resulted in elevated progesterone levels, a finding that is not surprising since progesterone is an allopregnanolone precursor and a pregnenolone metabolite, hence representing an intermediary neurosteroid in the biosynthetic pathway. Mean serum progesterone elevations to 2.17 ng/mL following adjunctive pregnenolone represent levels that are somewhat higher than typical male serum progesterone ranges defined by the commercially available radioimmunoassay kit utilized in this investigation (serum range for males 0.10-1.17 ng/mL), but less than or comparable to those achieved in human male brain (Lanthier & Patwardhan, 1986; Lacroix et al., 1987; Weill-Engerer et al., 2002). The current dosing strategy was tolerated without difficulty by all patients receiving pregnenolone (see summary of side effects in Table 8). Interestingly, evidence to date suggests that progesterone metabolism to allopregnanolone might represent a critical component contributing to several of progesterone's neuroprotective actions. For example, allopregnanolone is more effective than progesterone in diminishing cerebral cortical infarct volume following transient middle cerebral artery occlusion (Sayeed et al., 2006), and blocking progesterone metabolism to allopregnanolone prevents anticonvulsant effects attributed to progesterone (Kokate et al., 1999). It is thus possible that induction of this neurosteroid intermediary is relevant to pregnenolone mechanisms of action primarily because it is readily metabolized to the neuroprotective GABAergic neurosteroid allopregnanolone.

Possible Metabolism to DHEAS. DHEAS levels were significantly increased following pregnenolone administration, but the magnitude of this effect was relatively modest (approximately 16%). A prior investigation determined that DHEA administration significantly attenuated negative symptoms in patients with schizophrenia and resulted in concomitant elevations its sulfated derivative, DHEAS (Strous et al., 2003). It is therefore possible that pregnenolone metabolism to DHEAS is relevant to its therapeutic mechanism(s) of action.

Absence of Conversion to Other Steroids. Pregnenolone administration for eight weeks to patients with schizophrenia or schizoaffective disorder did not result in downstream elevations of cortisol, testosterone, free testosterone, DHEA, androstenedione, or estradiol (see Table 6). Since pregnenolone is a potential precursor to a number of steroids, pregnenolone could theoretically be metabolized to all of these molecules. The data presented herein suggest, however, that pregnenolone metabolism was weighted toward biosynthetic pathways resulting in pregnenolone sulfate and allopregnanolone formation, rather than toward glucocorticoid or sex steroid synthesis involving testosterone or estradiol.

Tolerability and Safety. Pregnenolone was very well-tolerated at the doses utilized in the randomized controlled trial disclosed herein. Side effects reported at greater frequency than placebo included two instances of mild restlessness, one instance of mild cold extremities, and one instance of mild muscle pain/stiffness. No patients receiving pregnenolone experienced a serious adverse event related to study medication. Patients randomized to pregnenolone did not demonstrate significant weight gain, blood pressure, or pulse rate changes during the course of this study. Glucose levels were unchanged, as were serum electrolyte panels, thyroid stimulating hormone, prolactin levels, and liver function tests.

However, cholesterol levels were significantly decreased following 8 weeks of treatment with pregnenolone. Interestingly, serum LDL levels were significantly decreased following adjunctive pregnenolone. As anticipated, pregnenolone serum levels at the completion of the study achieved or slightly exceeded pregnenolone levels observed in young adulthood (Morley et al., 1997).

Summary. Pregnenolone was very well-tolerated in this study and demonstrated no untoward effects on weight, blood pressure, pulse, glucose, prolactin, or other monitored laboratory parameters. Cholesterol and serum LDL levels were significantly decreased. Pregnenolone might thus represent a novel therapeutic advance for the treatment of hypercholesterolemia.

### REFERENCES

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
Addington et al. (1990) Schizophr Res 3: 247-251.
AO-Balboa et al. (2006) Proc Natl Acad Sci USA 103:14602-14607.
Akwa et al. (2001) Proc Natl Acad Sci USA 98: 14033-14037.
Andreasen (1983) Scale for the Assessment of Negative Symptoms (SANS) Iowa City: University of Iowa.
Anitschkow & Chalatov (1913) Zentralbl Allg Pathol 24:1-9.
Azcoitia et al. (2003) Neurobiol Aging 24: 853-860.
Barbaccia et al. (1996) Neuroendocrinology 63:166-172.
Barbaccia et al. (1998) Exp Gerontol 33:697-712.
Barbaccia et al. (2001) Neuropsychopharmacology 25: 489-497.
Barnes (1989) Br J Psychiatry 154: 672-676.
Belelli & Lambert (2005) Nature Reviews Neuroscience 6:565-575.
Benes & Berretta (2001) Neuropsychopharmacology 25: 1-27.
Benes et al. (2007) Proc Natl Acad Sci USA 104: 10164-10169.
Benitez-King et al. (2004) Curr Drug Targets CNS Neurol Disord 3: 515-533.
Berkow et al. (1997) The Merck Manual of Medical Information, Home ed., Merck Research Laboratories, Whitehouse Station, N.J.
Bowlby (1993) Mol Pharmacol 43: 813-819.
Brown & Goldstein (1983) Ann Rev Biochem 52:223-261.
Buchanan (2006) Am J Psychiatry 163: 1867-1869.
Buchanan (2007) Schizophr Bull 33: 1013-1022.
Buchanan et al. (2005) Schizophr Bull 31: 5-19.
Buchanan et al. (2007) Am J Psychiatry 164: 1593-1602.
Canadian Lipoprotein Conference Ad Hoc Committee on Guidelines for Dyslipoproteinemias (1990) Canada Med Assoc J 142:1371-1382.
Carter et al. (2008) Biol Psychiatry 64: 4-10.
Cascella et al. (1994) J Neural Transm Gen Sect 95: 105-111.
Charalampopoulos et al. (2004) Proc Natl Acad Sci USA 101: 8209-8214.
Charalampopoulos et al. (2005) Endocrinology 146: 3309-3318.
Charalampopoulos et al. (2006) Ann N Y Acad Sci 1088: 139-152.
Cheney et al. (1995) Neuroreport 6: 1697-1700.
Compagnone & Mellon (1998) Proc Natl Acad Sci USA 95:4678-4683.
Coyle (2006) Cell Mol Neurobiol 26: 365-384.
Danovich et al. (2008) Eur Neuropsychopharmacol 18: 24-33.
Darnaudery et al. (1998) J Neurochem 71: 2018-2022.
Darnaudery et al. (2002) Brain Res 951: 237-242.
Davison et al. (1950) Arch Intern Med 85: 365-389.
Dawson & Reid (1997) Nature 388: 235.
Debonnel et al. (1996) J Endocrinol 150:S33-S42.
Dickerson et al. (2007) Schizophr Res 93: 261-265.
Djebaili et al. (2004) Neuroscience 123: 349-359.
Djebaili et al. (2005) J Neurotrauma 22: 106-118.
Duch et al. (1998) Toxicol Lett 100-101:255-263.
Duncan et al. (2004) Schizophr Res 71: 239-248.
Ebadi (1998) CRC Desk Reference of Clinical Pharmacology. CRC Press, Boca Raton, Fla.
Ebner et al. (2006) Endocrinology 147: 179-190.
First et al. (1996) Structured clinical interview for DSM-IV axis I disorders-patient edition (SCID-I/P: version 2.0).
Flood et al. (1992) Proc Natl Acad Sci USA 89: 1567-1571.
Flood et al. (1995) Proc Natl Acad Sci USA 92: 10806-10810.
Fontaine-Lenoir et al. (2006) Proc Natl Acad Sci USA 103: 4711-4716.
Freeman et al. (1950) J Clin Endocrinol Metab 10: 1523-1532.
Freireich et al. (1966) Cancer Chemother Rep 50:219-244.
George et al. (1994) Biol Psychiatry 35: 775-780.
Ghoumari et al. (2003) J Neurochem 86: 848-859.
Glantz & Lewis (2000) Arch Gen Psychiatry 57: 65-73.
Glantz et al. (2006) Schizophr Res 81: 47-63.
Goff et al. (1995) Am J Psychiatry 152: 1213-1215.
Goff et al. (1996) Am J Psychiatry 153: 1628-1630.
Goff et al. (1999a) Arch Gen Psychiatry 56: 21-27.
Goff et al. (1999b) Biol Psychiatry 45: 512-514.
Goodman et al. (1996) Goodman & Gilman's the Pharmacological Basis of Therapeutics, 9th ed., McGraw-Hill Health Professions Division, New York.
Green (1996) Am J Psychiatry 153:321-330.
Green (2006) J Clin Psychiatry 67: e12.
Green et al. (2000) Schizophr Bull 26: 119-136.
Griffin et al. (2004) Nat Med 10: 704-711.
Grundy (1992) in Plasma Lipoproteins and Coronary Artery Disease: Role of Low-density Lipoproteins in Development of Coronary Artery Atherosclerosis. Kreisberg et al. (eds.) Blackwell Scientific: pp. 93-124.
Guest et al. (1950) J Am Med Assoc 143: 338-344.
Guidotti et al. (2005) Psychopharmacology (Berl) 180: 191-205.
Hakak et al. (2001) Proc Natl Acad Sci USA 98: 4746-4751.
Harvey et al. (2004) J Clin Psychiatry 65: 361-372.
He et al. (2004) Exp Neurol 189: 404-412.
Heinrichs et al. (1984) Schizophr Bull 10: 388-398.
Henderson et al. (1950) J Clin Endocrinol Metab 10: 455-474.
Heresco-Levy et al. (1999) Arch Gen Psychiatry 56: 29-36.
Heresco-Levy et al. (1996) Br J Psychiatry 169: 610-617.
Heresco-Levy et al. (2004) Biol Psychiatry 55: 165-171.
Heresco-Levy et al. (2005) Biol Psychiatry 57: 577-585.
Higashi et al. (2003a) J Pharm Biomed Anal 30: 1907-1917.
Higashi et al. (2003b) Biol Pharm Bull 26: 709-711.
Hsu et al. (2006) Nature 439: 480-483.
Ibanez et al. (2003) Prog Neurobiol 71: 49-56.
Imamura & Prasad (1998) Biochem Biophys Res Commun 243:771-775.
Irwin et al. (1994) J Pharmacol Exp Ther 271: 677-682.
Jarskog et al. (2005) Prog Neuropsychopharmacol Biol Psychiatry 29: 846-858.
Javitt (2004) Mol Psychiatry 9: 984-997: 979. Javitt (2007) Int Rev Neurobiol 78: 69-108.
Javitt et al. (2001) Int J Neuropsychopharmacol 4: 385-391.
Johansson et al. (2008) J Pharmacol Exp Ther 324: 759-768.
Jones et al. (2005) Eur J Neurosci 21: 1547-1554.
Katzung (2001) Basic & Clinical Pharmacology, 8th ed., Lange Medical Books/McGraw-Hill Medical Pub. Division, New York.
Kay et al. (1987) Schizophr Bull 13: 261-276.
Keefe et al. (2004) Schizophr Res 68: 283-297.
Keefe et al. (2007) Arch Gen Psychiatry 64: 633-647.
Keefe et al. (2008) Schizophr Res 102: 108-115.
Kirkpatrick et al. (2006) Schizophr Bull 32: 214-219.
Knight et al. (2007) Mol Psychiatry 12: 424-431.
Koenig et al. (1995) Science 268: 1500-1503.
Kokate et al. (1994) J Pharmacol Exp Ther 270: 1223-1229.
Kokate et al. (1996) Neuropharmacology 35: 1049-1056.
Kokate et al. (1999) J Pharmacol Exp Ther 288: 679-684.
Lacapere & Papadopoulos (2003) Steroids 68: 569-585.
Lacroix et al. (1987) J Steroid Biochem 28: 317-325.
Ladurelle et al. (2000) Brain Res 858: 371-379.
Lamond & Dawson (1999) J Sleep Res 8: 255-262.
Lanthier & Patwardhan (1986) J Steroid Biochem 25: 445-449.
Lencz et al. (2007) Mol Psychiatry 12: 572-580.
Lewis et al. (2003) Ann N Y Acad Sci 1003: 102-112.
Lewis et al. (2004) Psychopharmacology (Berl) 174: 143-150.
Lewis et al. (2005) Nat Rev Neurosci 6: 312-324.
Liere et al. (2004) J Lipid Res 45: 2287-2302.
Liu et al. (2003) Anal Chem 75: 5835-5846.
Lockhart et al. (2002) Neurosci Lett 328: 33-36.
Majewska et al. (1986) Science 232: 1004-1007.
Mao et al. (1983) Clin Chem 29:1890-1897.
Marder & Fenton (2004) Schizophr Res 72: 5-9.
Marder (2006) J Clin Psychiatry 67 Suppl 9: 31-35; discussion 36-42.
Marx et al. (2003) Neuropsychopharmacology 28: 1-13.
Marx et al. (2006a) Pharmacol Biochem Behav 84: 598-608.
Marx et al. (2006b) Pharmacol Biochem Behav 84: 609-617.
Marx et al. (2006c) Neuropsychopharmacology 31: 1249-1263.
Marx et al. (2006d) Psychopharmacology (Berl) 186: 462-472.
Marx et al. (2006e) Biol Psychiatry 60: 1287-1294.
Mathis et al. (1994) Psychopharmacology (Berl) 116: 201-206.
Mathis et al. (1996) Neuropharmacology 35: 1057-1064.
Mayo et al. (1993) Brain Res 607: 324-328.
Mayo et al. (2005) Neurobiol Aging 26: 103-114.
McGavack et al. (1951) J Clin Endocrinol Metab 11: 559-577.
Meieran et al. (2004) Psychoneuroendocrinology 29: 486-500.
Mellon & Griffin (2002) Trends Endocrinol Metab 13:35-43
Mellon et al. (2008) Brain Res Rev 57: 410-420.
Meziane et al. (1996) Psychopharmacology (Berl) 126: 323-330.
Millan (2005) Psychopharmacology (Berl) 179: 30-53.
Miller (1980) Ann Rev Med 31:97-108.
Morley et al. (1997) Proc Natl Acad Sci USA 94: 7537-7542.
Morrow et al. (1987) Eur J Pharmacol 142: 483-485.
Morrow et al. (1990) Mol Pharmacol 37: 263-270.
Morrow et al. (1995) Ann NY Acad Sci 771:257-272.
Mtchedlishvili & Kapur (2003) Mol Pharmacol 64: 857-864.
Murakami et al. (2000) Proc Natl Acad Sci USA 97: 3579-3584.
National Cholesterol Education Program (2002) Circulation 106:1333-1445.
Naylor et al. (2008) J Clin Endocrinol Metab 93: 3173-3178.
Nuechterlein & Green (2006) MATRICS Consensus Cognitive Battery (MCCB) Los Angeles: MATRICS Assessments Inc.
Nuechterlein et al. (2008) Am J Psychiatry 165: 203-213.
Pallares et al. (1998) Neuroscience 87: 551-558.
Park-Chung et al. (1999) Brain Res 830:72-87.
Patton et al. (1983) Clin Chem 29:1898-1903.
Paul & Purdy (1992) FASEB J 6:2311-2322.
PCT International Publication No. WO 93/25521.
Pincus & Hoagland (1944) J Aviation Med 15: 98-115.
Pincus & Hoagland (1945a) Psychosomatic Medicine 7: 342-346.
Pincus & Hoagland (1945b) II. Psychosomatic Medicine 7: 347-352.
Pinna et al. (2006) Psychopharmacology (Berl) 186: 362-372.
Porcu et al. (2003) Pharmacol Biochem Behav 74:683-690.
Porcu et al. (2008) Psychoneuroendocrinology 33: 214-226.
Purdy et al. (1991) Proc Natl Acad Sci USA 88:4553-4557.
Remington et al. (1975) Remington's Pharmaceutical Sciences, 15th ed., Mack Pub. Co., Easton, Pa.
Roberts (1995) Biochem Pharmacol 49: 1-16.
Robertson et al. (2006) Exp Neurol 197: 235-243.
Romeo et al. (1994) J Pharmacol Exp Ther 270: 89-96.
Roof et al. (1997) Mol Chem Neuropathol 31: 1-11.
Rosse et al. (1996) Clin Neuropharmacol 19: 444-450.
Rujescu et al. (2006) Biol Psychiatry 59: 721-729.
Rupprecht & Holsboer (1999) Trends Neurosci 22:410-416.
Sakamoto et al. (2001) J Neurosci 21: 6221-6232.
Sayeed et al. (2006) Ann Emerg Med 47: 381-389.
Schumacher et al. (2000) J Neurocytol 29: 307-326.
Schumacher et al. (2008) Neurochem Int 52: 522-540.
Sepehry et al. (2007) J Clin Psychiatry 68: 604-610.
Serra et al. (2001) Psychopharmacology (Berl) 158: 48-54.
Shephered et al. (1987) Br Med J 295:1245-1246.
Simpson & Angus (1970) Acta Psychiatr Scand Suppl 212: 11-19.
Sliwinski et al. (2004) J Neurosci Res 78: 691-701.
Speight et al. (1997) Avery's Drug Treatment: A Guide to the Properties, Choice, Therapeutic Use and Economic Value of Drugs in Disease Management, 4th ed. Adis International, Auckland/Philadelphia.
Stein (2008) Brain Res Rev 57: 386-397.
Strous et al. (2003) Arch Gen Psychiatry 60: 133-141.
Study Group of the European Atherosclerosis Society (1988) Eur Heart J 9:571-600.
Tkachev et al. (2007) Int J Neuropsychopharmacol 10: 557-563.
Tsai et al. (1998) Biol Psychiatry 44: 1081-1089.
Tsai et al. (1999) Am J Psychiatry 156: 1822-1825.
U.S. Patent Application Publication No. 20060188558.
U.S. Pat. Nos. 3,598,122; 5,016,652; 5,326,902; 5,234,933; 5,935,975; 6,106,856; 6,162,459; 6,180,082; 6,495,605; 6,582,724.
Vallee et al. (1997) Proc Natl Acad Sci USA 94: 14865-14870.
Vallee et al. (2000) Anal Biochem 287:153-166.
Vallee et al. (2001) Eur J Neurosci 14: 2003-2010.
Vallee et al. (2003) J Steroid Biochem Mol Biol 85: 329-335.
van Berckel et al. (1996) Biol Psychiatry 40: 1298-1300.
VanLandingham et al. (2007) Neurosci Lett 425: 94-98.
Wang et al. (1997) J Steroid Biochem Mol Biol 62: 299-306.
Wang et al. (2005) J Neurosci 25: 4706-4718.
Weill-Engerer et al. (2002) J Clin Endocrinol Metab 87: 5138-5143.
Wieland et al. (1991) Brain Res 565:263-268.
Wright et al. (2007) Ann Emerg Med 49: 391-402: 402 e391-392.
Wu et al. (1991) Mol Pharmacol 40: 333-336.
Xiao et al. (2008) Grit Care 12: R61.
Xilouri & Papazafiri (2006) Eur J Neurosci 23: 43-54.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for ameliorating a symptom associated with hypercholesterolemia, hyperlipidemia, or both in a subject, the method comprising administering to a subject in need thereof an effective amount of a composition comprising pregnenolone (PG), allopregnanolone (ALLO), dehydroepiandrosterone (DHEA), progesterone (PROG), precursors thereof, metabolites thereof, pharmaceutically acceptable salts thereof, derivatives thereof, or combinations thereof.

2. The method of claim 1, wherein the composition is administered in a sustained release formulation, a controlled release formulation, or a combination thereof.

3. The method of claim 2, wherein the sustained release formulation, the controlled release formulation, or the combination thereof is selected from the group consisting of an oral formulation, a peroral formulation, a buccal formulation, an enteral formulation, a pulmonary formulation, a rectal formulation, a vaginal formulation, a nasal formulation, a lingual formulation, a sublingual formulation, an intravenous formulation, an intraarterial formulation, an intracardial formulation, an intramuscular formulation, an intraperitoneal formulation, a transdermal formulation, an intracranial formulation, an intracutaneous formulation, a subcutaneous formulation, an aerosolized formulation, an ocular formulation, an implantable formulation, a depot injection formulation, and combinations thereof.

4. The method of claim 1, wherein the composition comprises at least two active agents selected from the group consisting of PG, ALLO, DHEA, PROG, precursors thereof, metabolites thereof, pharmaceutically acceptable salts thereof, derivatives thereof, or combinations thereof.

5. The method of claim 1, wherein the effective amount is sufficient to lower blood cholesterol, lower blood low density lipoprotein (LDL), raise blood high density lipoprotein (HDL), or combinations thereof in the subject.

6. The method of claim 1, wherein the derivative comprises a sulfated derivative.

7. The method of claim 1, further comprising administering to the subject at least one additional cholesterol lowering composition, wherein the at least one additional cholesterol lowering composition is administered to the subject before, after, and/or at the same time as the composition comprising PG, ALLO, DHEA, PROG, precursors thereof, metabolites thereof, pharmaceutically acceptable salts thereof, derivatives thereof, or combinations thereof.

8. A method for ameliorating at least one symptom resulting from undesirable cholesterol biosynthesis in a subject, the method comprising administering to a subject in need thereof an effective amount of a composition comprising pregnenolone (PG), allopregnanolone (ALLO), dehydroepiandrosterone (DH EA), progesterone (PROG), precursors thereof, metabolites thereof, pharmaceutically acceptable salts thereof, derivatives thereof, or combinations thereof.

9. The method of claim 8, wherein the composition is administered in a sustained release formulation, a controlled release formulation, or a combination thereof.

10. The method of claim 9, wherein the sustained release formulation, the controlled release formulation, or the combination thereof is selected from the group consisting of an oral formulation, a peroral formulation, a buccal formulation, an enteral formulation, a pulmonary formulation, a rectal formulation, a vaginal formulation, a nasal formulation, a lingual formulation, a sublingual formulation, an intravenous formulation, an intraarterial formulation, an intracardial formulation, an intramuscular formulation, an intraperitoneal formulation, a transdermal formulation, an intracranial formulation, an intracutaneous formulation, a subcutaneous formulation, an aerosolized formulation, an ocular formulation, an implantable formulation, a depot injection formulation, and combinations thereof.

11. The method of claim 8, wherein the undesirable cholesterol biosynthesis in subject results in hypercholesterolemia, hyperlipidemia, or a combination thereof in the subject.

12. The method of claim 11, wherein the subject also has a neuropsychiatric disorder.

13. The method of claim 12, wherein the neuropsychiatric disorder is selected from the group consisting of a psychotic disorder, a cognitive disorder, a neurodegenerative disorder, an anxiety disorder, a pain disorder, and combinations thereof.

14. The method of claim 12, wherein the neuropsychiatric disorder is selected from the group consisting of schizophrenia, schizoaffective disorder, Alzheimer's disease, Attention Deficit Disorder/Attention Deficit Hyperactivity Disorder, depression, bipolar disorder, post-traumatic stress disorder (PTSD), alcohol abuse, alcohol dependence, drug dependence, drug abuse, and combinations thereof.

15. The method of claim 8, wherein the composition comprises at least two active agents selected from the group consisting of PG, ALLO, DHEA, PROG, precursors thereof, metabolites thereof, pharmaceutically acceptable salts thereof, derivatives thereof, or combinations thereof.

16. The method of claim 8, wherein the effective amount is sufficient to lower blood cholesterol, lower blood low density lipoprotein (LDL), raise blood high density lipoprotein (HDL), or combinations thereof in the subject.

17. The method of either of claims 1 and 8, wherein the administering causes substantially no decrease in high density lipoprotein (HDL) levels in the blood of the subject.

18. A method for lowering cholesterol, low density lipoprotein, or both in the serum of a subject, the method comprising administering to a subject in need thereof an effective amount of a composition comprising pregnenolone (PG), allopregnanolone (ALLO), dehydroepiandrosterone (DHEA), progesterone (PROG), precursors thereof, metabolites thereof, pharmaceutically acceptable salts thereof, derivatives thereof, or combinations thereof.

19. The method of claim 18, wherein the composition comprises PG, a sulfated derivative of PG, a precursor of PG, a metabolite of PG, a pharmaceutically acceptable salt of PG, or a combination thereof.

20. The method of claim 18, wherein the administering causes substantially no decrease in high density lipoprotein (HDL) levels in the blood of the subject.

21. The method of any of claims 1, 8, and 18, further comprising administering to the subject an additional pharmaceutical composition comprising an active agent selected from the group consisting of a cholesterol lowering agent, an LDL reducing agent, an HDL increasing agent, or a combination thereof.
